(19) United States
(12) Patent Application Publication
(10) Pub. No.: US 2018/0201694 A1
(43) Pub. Date: Jul. 19, 2018

(54) CHITIN DERIVATIVES, METHOD FOR PRODUCTION AND USES THEREOF

(71) Applicant: JAWAHARLAL NEHRU CENTRE FOR ADVANCED SCIENTIFIC RESEARCH (JNCASR), Bangalore (IN)

(72) Inventors: Jayanta Haldar, Bangalore (IN); Jiaul Hoque, Bangalore (IN); Goutham Belagula Manjunath, Bangalore (IN); Padma Akkapeddi, Bangalore (IN)

(21) Appl. No.: 15/105,153

(22) PCT Filed: Dec. 16, 2014
(86) PCT No.: PCT/IB2014/002788

§ 371 Date: Jun. 16, 2016

(30) Foreign Application Priority Data
Dec. 17, 2013 (IN) 5893/CHE/2013

(51) Int. Cl. C08B 37/08; A01N 43/16; C09D 5/14; C09D 105/08; A61K 31/722; A61P 31/04; A61P 31/10; A61P 31/12; A01N 59/16; A61L 15/28; A61L 15/44; A61L 26/00; A61L 27/20; A61L 27/34; A61L 27/54; A61L 31/10; A61L 31/16; A61L 31/04; A61L 29/16; A61L 29/08; A61L 29/04

(52) U.S. Cl. CPC C08B 37/003; A61L 2300/606; C09D 5/14; C09D 105/08; A61K 31/722; A61P 31/04; A61P 31/10; A61P 31/12; A01N 59/16; A61L 15/28; A61L 15/44; A61L 26/0023; A61L 26/0066; A61L 27/20; A61L 27/34; A61L 27/54; A61L 31/10; A61L 31/16; A61L 31/042; A61L 29/16; A61L 29/085; A61L 29/043; A61L 2300/404; A01N 43/16

(57) ABSTRACT

The present disclosure relates to chitin derivatives of Formula (I), its isomers, prodrugs and pharmaceutically acceptable salts thereof. The present disclosure further relates to a process of preparing the chitin derivatives, its isomers, prodrugs and pharmaceutically acceptable salts thereof.

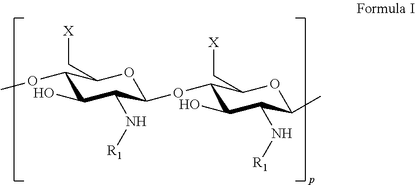

The compounds of the present disclosure are useful in antimicrobial coatings. The present disclosure further relates to an antibacterial polymeric nanocomposite and a process for preparing the antibacterial polymeric nanocomposites.

### TECHNICAL FIELD

The present disclosure relates to chitin derivatives, its isomers, prodrugs and pharmaceutically acceptable salts thereof. The present disclosure further relates to a process of preparing the chitin derivatives, its isomers, prodrugs and pharmaceutically acceptable salts thereof. The present disclosure also relates to compositions and methods of preventing conditions and diseases that are caused by microorganism.

The present disclosure further relates to an antibacterial polymeric nanocomposite and a process for preparing the antibacterial polymeric nanocomposites. The present disclosure also relates to nanocompositions and methods of preventing conditions and diseases caused by microorganisms.

### BACKGROUND

Bacterial contamination is a growing threat in medical clinics, operating rooms and public settings. High-touch surfaces or commonly touched surfaces are generally contaminated by bacteria transferred from people and surfaces. Usually ordinary materials are not antimicrobial and their modification is required in order to prevent infections. Surfaces chemically modified with polyethylene glycol and certain other synthetic polymers can repel, although not kill, microorganisms. Alternatively, materials can be impregnated with antimicrobial agents, such as antibiotics, metal or metal oxide nanoparticles, quaternary ammonium compounds, or iodine, which are gradually released into the surrounding medium over time and kill microorganisms. Although these strategies have been verified in aqueous solutions containing bacteria, they would not be expected to be effective against airborne bacteria in the absence of a liquid medium. This is especially true for release-based materials, which may also be liable to become impotent when the leaching antibacterial agent is exhausted.

Despite sterilization and cleansing, a variety of materials in the medical environment can retain dangerous organisms trapped in a biofilm, hence to be passed on to other hosts. For procedures involving implantable medical devices, avoiding infection can be particularly problematic because bacteria can develop into biofilms, which protect the microbes from clearing by the subject's immune system and from the action of drugs. As these infections are difficult to treat with antibiotics, removal of the device is often necessitated, which can be traumatic to the patient and increase the medical cost. In addition, hospital-acquired infections are more likely to involve organisms that have developed resistance to a number of antibiotics thus making them difficult to treat. Thus, there is a keen interest in materials capable of killing harmful microorganism, especially materials that could be used to coat surfaces of common objects, medical devices and implants, etc. to render them antiseptic and thus unable to transmit infections caused by the microorganism.

Chitin, the second most abundant naturally occurring polymer, is inherently antimicrobial. But the insolubility of the polymer in almost all the common organic solvents limits its practical use as antimicrobial coatings. Moreover, the antimicrobial activity of the pristine chitin is very low. Furthermore, developments of antimicrobial coatings are deeply restricted by the use of the synthetic polymers which are non-biocompatible and non-biodegradable in nature which limit their in-vivo applications. In general, the coating formulations involve covalent modifications of the surfaces further limits the practical usage of the coatings as it requires several harsh and synthetic chemical reactions.

U.S. Pat. No. 7,838,643B1 relates to novel quaternized polymers, especially of chitin/chitosan type, and to carbohydrate polymers carrying quaternized ammonium groups, especially piperazinium groups.

U.S. Pat. No. 6,306,835B1 relates to 3-trimethylammonium-2-hydroxypropyl-N-chitosan (CHI-Q188) and related chitosan derivatives exhibit antimicrobial activity at concentrations as low as 10-20 μg/mL, has been reported to exhibit antimicrobial activity.

Bazito and coworkers synthesized sugar-based cationic surfactants and examined the effect of increasing the length of the hydrophobic moiety on aggregate formation in water (Journal of Surfactants and Detergents, 395-400, 4, 2001).

Any agent used to prevent infection or to impair biofilm formation in the medical environment must be non-toxic towards mammalian cells and safe to the environment. Certain biocidal agents, in quantities sufficient to interfere with biofilms, can damage host tissues. Antibiotics introduced into local tissue areas can induce the formation of resistant organisms which can then form biofilm communities whose planktonic microorganisms would likewise be resistant to the particular antibiotics. Furthermore, long term systemic antibiotic therapy to eradicate infection causes increased level of toxicity towards host cell. Moreover, any anti-biofilm or antifouling agent must not interfere with the salubrious characteristics of a medical device. Thus there is a need to identify and/or develop new compounds and/or derivatives that has enhanced activity against bacterial strains including multidrug resistant bacteria while the compounds are non-toxic and biodegradable in nature.

### SUMMARY

The present disclosure relates to a compound of Formula I

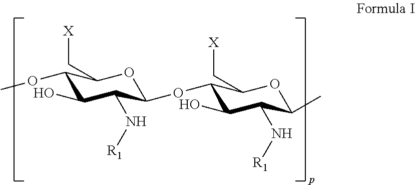

wherein:

### X is

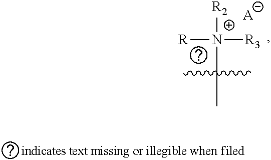

OH and combinations thereof;
R₂, R₃ and R₄ are independently selected from the group consisting of hydrogen, substituted or unsubstituted C₁₋₂₂ alkyl, substituted or unsubstituted C₆₋₁₀ aryl,

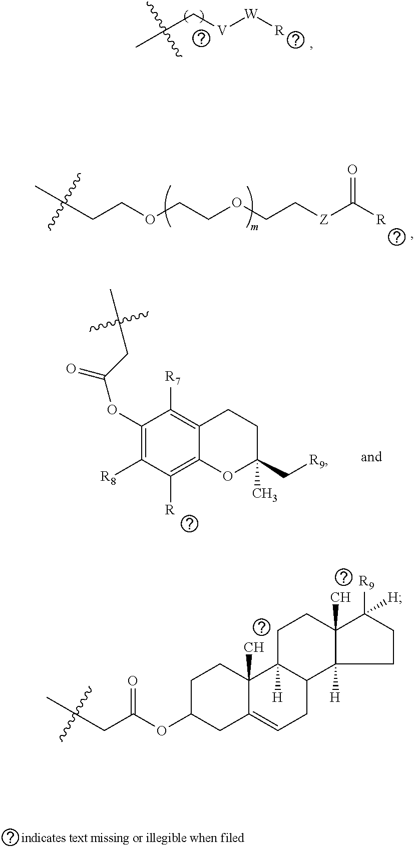

or
R₂ and R₃ taken together to form a substituted or unsubstituted cyclic ring system which is saturated or partially unsaturated and optionally have additional heteroatoms selected from O, N or S; or
R₂ and R₃ taken together to form a substituted or unsubstituted aromatic ring system optionally having heteroatoms selected from O, N or S; or
R₂, R₃ and R₄ may combine to form a substituted or unsubstituted bicylic ring system which is saturated, partially unsaturated or fully unsaturated, a substituted or unsubstituted aromatic ring system-and-optionally having heteroatoms selected from O, N or S;
V and W are independently selected from the group consisting of O, NH and —CO;

### Z is O or —NH;

R₁ is selected from the group consisting of hydrogen, C₁₋₁₆ alkyl, C₆₋₁₀ aryl, —COR₁₀, and combinations thereof;
R₅ and R₉ are independently selected from the group consisting of hydrogen, substituted or unsubstituted C₁₋₁₆ alkyl, substituted or unsubstituted C₂₋₂₄ alkenyl, substituted or unsubstituted C₆₋₁₀ aryl, and combinations thereof;
R₆, R₇ and R₈ are independently selected from hydrogen and methyl;
A^{⊖} is negatively charged counter anion;
R₁₀ is selected from the group consisting of C₁₋₁₆ alkyl and C₆₋₁₀ aryl, wherein alkyl and aryl are optionally substituted with halogen, alkyl, and aryl;
l is 0 to 4;
m is 0 to 3; and
p is 1 to 1000, wherein the degree of substitution of R₁ with hydrogen, C₁₋₁₆ alkyl, C₆₋₁₀ aryl, or —COR₁₀ in the compound of formula I is in the range of 20-100%; and the degree of substitution of X with

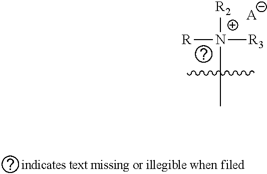

in the compound of Formula I is in the range of 10-90%.

The present disclosure further relates to a compound of Formula I, for use in antimicrobial coatings.

The present disclosure relates to a pharmaceutical composition comprising a compound of Formula I, optionally in combination with one or more other pharmaceutical compositions.

The present disclosure relates to a method of preparing biodegradable antimicrobial coatings and/or surfaces with or without pharmaceutical compositions.

The present disclosure further relates to an article comprising a substrate, wherein the substrate is coated with or impregnated with the composition comprising the compound of Formula I, or the pharmaceutically acceptable salt.

The present disclosure relates to a process for preparation of compound of Formula I.

The present disclosure further relates to an antibacterial polymeric nanocomposite and a process for preparing the antibacterial polymeric nanocomposites.

The present disclosure further relates to an article comprising a substrate, wherein the substrate is coated with or impregnated with the composition comprising the polymeric nanocomposites.

These and other features, aspects, and advantages of the present subject matter will become better understood with reference to the following description. This summary is provided to introduce a selection of concepts in a simplified form. This summary is not intended to identify key features or essential features of the disclosure, nor is it intended to be used to limit the scope of the subject matter.

### BRIEF DESCRIPTION OF DRAWINGS

The detailed description is described with reference to the accompanying figures. In the figures, the left-most digit(s) of a reference number identifies the figure in which the reference number first appears. The same numbers are used throughout the drawings to reference like features and components.

FIG. 1 illustrates antibacterial activity of the compounds of formula I (1b-1c, 2a-2e and 3a-3c) against S. aureus (FIG. 1A) and E. coli (FIG. 1B).

FIG. 2 illustrates the kinetics of the antibacterial activity of the compounds of formula I (1c and 2c) at different concentrations against S. aureus (FIG. 2A) and E. coli (FIG. 2B).

FIG. 3 illustrates the antibacterial activity of the compound of formula I (2c) coated, glass slides against S. aureus (Figure A-E) and E. coli (Figure F-J) by spray method. Figure A and F illustrate non-coated glass slides (controls); Figure B and G illustrate 2c coated slides with 4 μg/cm²; Figure C and H illustrate 2c coated slides with 8 μg/cm²; Figure D and I illustrate 2c coated slides with 16 μg/cm²; Figure E and J illustrate 2c coated slides with 32 μg/cm².

FIG. 4 illustrates the antibacterial activity of the compound of Formula I (2c) along with polylactic acid (PLA) coated glass slides against S. aureus (Figure A-D) and E. coli (Figure E-H) by spray method. Figure A and D illustrate glass slides coated only with PLA (255 μg/cm²); Figure B; C and D illustrate (PLA+2c) coated slides with (255+4) μg/cm², (255+8) μg/cm² and (255+16) μg/cm² respectively; Figure F, G and H illustrate (PLA+2c) coated slides with (255+8) μg/cm², (255+16) μg/cm² and (255+32) μg/cm² respectively.

FIG. 5 illustrates the cytoplasmic membrane depolarization ability of the compound of Formula I (2c) at 200 μg/mL against S. aureus (Figure A) and at 2000 μg/mL against E. coli respectively (Figure B); and intracellular potassium ion leakage ability of chitin derivatives at 200 μg/mL against S. aureus (Figure C) and at 2000 μg/mL against E. coli respectively (Figure D).

FIG. 6 illustrates the fluorescence microscopy images of S. aureus (A and B) and E. coli (C and D) cells after a 4 h exposure to the uncoated surfaces (A and C) and surfaces coated with the compound of Formula I (2c) (B and D). Live (A and C) and dead cells (B and D) were stained with staining agents SYTO 9 and propidium iodide (PI) respectively. Scale bar 20 μM.

FIG. 7 illustrates the scanning electron microscopy (SEM) images of S. aureus (Figure A and B) and E. coli (Figure C and D) cells after a 2 h exposure to the uncoated surfaces (Figure A and C) and surfaces coated with the compound of Formula I (2c) (Figure B and D).

FIG. 8 illustrates the hemolytic activity of the compounds of Formula I (1b-1c, 2a-2c and 3a-3c) against human RBC measured by the release of hemoglobin from the lysed RBC.

FIG. 9 illustrates the optical microscopy images of HEK 293 cell line: Figure A illustrates cells grown over non-coated surface showing normal morphology; Figure B. and C illustrate cells grown over the compound of Formula I (2c) coated surfaces (10 μg/mL and 156 μg/mL respectively) showing retained morphology; Figure D illustrates triton-X treated cells. Scale bar 20 μM.

FIG. 10 illustrates the SEM images of films of the compound of Formula I (2c): Figure A shows an image of the film after coating; Figure B shows an image of the film after incubation with only buffer for 20 days; Figure C shows an image of the film after incubation with lysozyme in buffer solution for 15 days and Figure D shows an image of the film after incubation with lysozyme in buffer solution for 20 days. Scale bar 5 μM.

FIG. 11 illustrates the UV-visible absorption spectrum (FIG. 11A) and transmission electron microscopy (TEM) images (FIG. 11B) of silver nanoparticles formed in-situ from 1:0.5 (1c: AgPTS) mixture.

FIG. 12 illustrates the antibacterial activity of the nanocomposite (1:0.5) coated glass surfaces against S. aureus (A-C) and E. coli (D-F) respectively by spray method: Figure A and D non-coated glass slides (controls); Figure B and E slides coated with 1c (30 and 60 μg/cm² respectively); Figure C and F slides coated with the nanocomposite ((10+5) μg/cm² and (20+10) μg/cm² respectively).

FIG. 13 illustrates the antibacterial activity of the nanocomposite (1:0.5): minimum inhibitory concentrations (MICs) of the nanocomposite along with the polymer 1c and AgPTS against S. aureus (FIG. 13A) and E. coli (FIG. 13B).

FIG. 14 illustrates the kinetics of the antibacterial activity of the compound of Formula I (1c), AgPTS and the nanocomposite (1:0.5 1c: AgPTS) at two diffeirent concentrations: MIC and 6×MIC towards S. aureus.

### DETAILED DESCRIPTION

In the structural formulae given herein and throughout the present disclosure, the following terms have been indicated meaning, unless specifically stated otherwise.

### Definitions

The term “alkyl” refers to a monoradical branched or unbranched saturated hydrocarbon chain having from 1 to 22 carbon atoms. This term is exemplified by groups such as methyl, ethyl, n-propyl, iso-propyl, n-butyl, iso-butyl, t-butyl, n-hexyl, n-decyl, tetradecyl, and the like. By way of further example, a C₁-C₂₀ alkyl contains at least one but no more than 20 carbon atoms. A methyl group (i.e., CH₃—) is an example of a C₁ alkyl radical. A dodecyl group (i.e., CH₃(CH₂)₁₂—) is an example of a C₁₂ alkyl radical.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This polymers described herein are not intended to be limited in any manner by the permissible substituents of organic compounds.

The term “substituted alkyl” refers to an alkyl group as defined above, having 1 to 10 substituents, selected from the group consisting of hydroxyl, alkyl, aryl, alkoxy, halogen, haloalkyl, perhaloalhyl, cyano, or keto;

The term “alkenyl” refers to a monoradical of a branched or unbranched unsaturated hydrocarbon group preferably having from 2 to 24 carbon atoms, and having 1, 2, 3, 4, 5 or 6 double bonds. Preferred alkenyl groups include ethenyl or vinyl (—CH═CH₂), 1-propylene or allyl (—CH₂CH═CH₂), isopropylene (—C(CH₃)═CH₂), bicyclo [2.2. 1] heptene, octadec-9-enyl radical (CH₃(CH₂)₇CH═CH(CH₂)₇CH₂—), which is a C₁₈ aliphatic radical comprising single alkenyl group and octadec-9,12-dienyl radical (CH₃(CH₂)₄CH═CHCH₂CH(CH₂)₇CH₂—), which is a C₁₈ aliphatic radical comprising two alkenyl groups. Further examples of aliphatic radicals include allyl (CH₂═CHCH₂—), propargyl (CH≡CCH₂—), aminocarbonyl (i.e., —CONH₂), carbonyl, 2,2-dicyanoisopropylidene (i.e., —CH₂C(CN)₂CH₂—), methyl (i.e., —CH₃), methylene (i.e., —CH₂—), ethyl, ethylene, formyl (i.e. —CHO), hexyl, hexamethylene, hydroxymethyl (i.e. —CH₂OH), mercaptomethyl (i.e., —CH₂SH), methylthio (i.e., —SCH₃), methylthiomethyl (i.e., —CH₂SCH₃), methoxy, methoxycarbonyl (i.e., CH₃OCO—), nitromethyl (i.e., —CH₂NO₂), thiocarbonyl, trimethylsilyl (i.e., (CH₃)₃Si—), t-butyldimethylsilyl, 3-trimethyoxysilypropyl (i.e., (CH₃O)₃SiCH₂CH₂CH₂—), vinyl, vinylidene, and the like.

The term “substituted alkenyl” refers to an alkenyl group as defined above having 1, or 2 substituents, selected from the group consisting of hydroxyl, alkyl, aryl, alkoxy, halogen, haloalkyl, perhaloalhyl, cyano, or keto;

“Halo” or “Halogen”, alone or in combination with any other term means halogens such as chloro (Cl), fluoro (F), bromo (Br) and iodo (I).

“Haloalkyl” refers to a straight chain or branched chain haloalkyl group. The alkyl group may be partly or totally halogenated. Representative examples of haloalkyl groups include but are not limited to fluoromethyl, chloromethyl, bromomethyl, difluoromethyl, dichloromethyl, dibromomethyl, trifluoromethyl, trichloromethyl, 2-fluoroethyl, 2-chloroethyl, 2-bromoethyl, 2,2,2-trifluoroethyl, 3-fluoropropyl, 3-chloropropyl, 3-bromopropyl and the like.

The term “aryl” refers to an aromatic carbocyclic group of 6 to 10 carbon atoms having a single ring or multiple rings, or multiple condensed (fused) rings.

The term “substituted aryl” refers to an alkynyl group as defined above having 1 to 4 substituents, selected from the group consisting of hydroxyl, alkyl, aryl, alkoxy, halogen, haloalkyl, perhaloalhyl, cyano, or keto;

The term “arylalkyl” refers to an aryl group covalently linked to an alkylene group, where aryl and alkylene are defined herein.

As used herein, the term “aromatic radical” includes but is not limited to phenyl, pyridyl, furanyl, thienyl, naphthyl, phenylene, and biphenyl radicals. As noted, the aromatic radical contains at least one aromatic group. The aromatic group it invariably a cyclic structure having 4n+2 “delocalized” electrons where “n” is an integer equal to 1 or greater, as illustrated by phenyl groups (n=1), thienyl groups (n=1), furanyl groups (n=1), naphthyl groups (n=2), azulenyl groups (n=2), and anthraceneyl groups (n=3). The aromatic radical may also include nonaromatic components. For example, benzyl (C₆H₅CH₂—), naphthyl-1-methyl (C₁₀H₇CH₂—), anthracenyl-1-methyl (C₁₄H₉CH₂—) are aromatic radicals, which comprise a phenyl ring, a naphthyl ring, an anthracenyl ring (the aromatic group) respectively and a methylene group (the nonaromatic component). Similarly a tetrahydronaphthyl radical is an aromatic radical comprising an aromatic group (C₆H₃) fused to a nonaromatic component —(CH₂)₄—. For convenience, the term “aromatic radical” is defined herein to encompass a wide range of functional groups such as alkyl groups, alkenyl groups, alkynyl groups, haloalkyl groups, haloaromatic groups, conjugated dienyl groups, alcohol groups, ether groups, aldehyde groups, ketone groups, carboxylic acid groups, acyl groups (for example carboxylic acid derivatives such as esters and amides), amine groups, nitro groups, and the like. For example, the 4-methylphenyl radical is a C₇ aromatic radical comprising a methyl group, the methyl group being a functional group which is an alkyl group. Similarly, the 2-nitrophenyl group is a C₆ aromatic radical comprising a nitro group, the nitro group being a functional group. Aromatic radicals include halogenated aromatic radicals such as 4-trifluoromethylphenyl, hexafluoroisopropylidenebis(4-phen-1-yloxy) (i.e., —OPhC(CF₃)₂PhO—), 4-chloromethylphen-1-yl, 3-trifluorovinyl-2-thienyl, 3-trichloromethylphen-1-yl (i.e., 3-CCl₃Ph-), 4-(3-bromoprop-1-yl)phen-1-yl (i.e., 4-BrCH₂CH₂CH₂Ph-), and the like. Examples of aromatic radical include but are not limited to, tocopherol and tocotrienol. Further examples of aromatic radicals include 4-allyloxyphen-1-oxy, 4-aminophen-1-yl (i.e., 4-H₂NPh-), 3-aminocarbonylphen-1-yl (i.e., NH₂COPh-), 4-benzoylphen-1-yl, dicyanomethylidenebis(4-phen-1-yloxy) (i.e., —OPhC(CN)₂PhO—), 3-methylphen-1-yl, methylenebis(4-phen-1-yloxy) (i.e., —OPhCH₂PhO—), 2-ethylphen-1-yl, phenylethenyl, 3-formyl-2-thienyl, 2-hexyl-5-furanyl, hexamethylene-1,6-bis(4-phen-1-yloxy) (i.e., —OPh(CH₂)₆PhO—), 4-hydroxymethylphen-1-yl i.e., 4-HOCH₂Ph-), 4-mercaptomethylphen-1-yl (i.e., 4-HSCH₂Ph-; 4-methylthiophen-1-yl (i.e., 4-CH₃SPh-), 3-methoxyphen-1-yl, 2-methoxycarbonylphen-1-yloxy (e.g. methyl salicyl), 2-nitromethylphen-1-yl (i.e., 2-NO₂CH₂Ph), 3-trimethylsilylphen-1-yl, 4-t-butyldimethylsilylphenl-1-yl, 4-vinylphen-1-yl, vinylidenebis (phenyl), and the like. The term “a C₃-C₁₀ aromatic radical” includes aromatic radicals containing at least three but no more than 10 carbon atoms. The aromatic radical 1-imidazolyl (C₃H₂N₂—) represents a C₃ aromatic radical. The benzyl radical (C₇H₇—) represents a C₇ aromatic radical.

The term “cycloalkyl” refers to carbocyclic groups of from 3 to 22 carbon atoms having a single cyclic ring which may be substituted and partially unsaturated or multiple condensed rings which may be substituted and partially unsaturated.

“Cycloalkylalkyl” refers to an alkyl radical as defined above which is substituted by a cycloalkyl radical as defined above. Representative examples of cycloalkyldlkyl include but are not limited to cyclopropylmethyl, cyclobutylmethyl, cyclopentylmethyl, cyclohexylmethyl, 1-cyclopentylethyl, 1-cyclohexylethyl, 2-cyclopentylethyl, 2-cyclohexylethyl, cyclobutylpropyl, cyclopentylpropyl, cyclohexylbutyl and the like.

The term “heterocyclyl” refers to a saturated or partially unsaturated group having a single ring or multiple condensed rings, having from 1 to 40 carbon atoms selected from nitrogen, sulfur, and/or oxygen within the ring. Heterocyclic groups can have a single ring or multiple condensed rings.

The term “heterocyclylalkyl” refers to a heterocyclyl group covalently linked to an alkylene group, where heterocyclyl and alkylene are defined herein.

The term “heteroaryl” refers to an aromatic cyclic group having 6 to 10 carbon atoms and having heteroatoms selected from oxygen, nitrogen and sulfur within at least one ring (if there is more than one ring). Such heteroaryl groups can have a single ring (e.g. pyridyl or furyl) or multiple condensed rings (e.g. indolizinyl, benzothiazolyl, or benzothienyl).

The terms “hydrophilic” and “hydrophobic” are art-recognized and mean water-loving and water-hating, respectively. In general, a hydrophilic substance will dissolve in water, and a hydrophobic one will not. The term “hydrophobic” as used herein to describe a compound of the present disclosure or a substituent thereon, refers to the tendency of the compound or substituent thereon to lack an affinity for, to repel or to fail to absorb water, or to be immiscible in water. The term “hydrophobic” is not meant to exclude compounds or substituents thereon that are not completely immiscible in water.

The term “water insoluble” as generally used herein means that the polymer has a solubility of less than approximately 0.1% (w/w) in water under standard conditions at room temperature or body temperature.

The term “pharmaceutically acceptable salt” as used herein, refers to salts of the compounds that are substantially non-toxic to living organisms such that it could be effectively used to prevent or treat the infections. Typical pharmaceutically acceptable salts of the compounds of the subject invention include those salts, which are prepared by reaction of the compounds of the present invention with a pharmaceutically acceptable mineral acid or organic acid. Such salts are classified as acid addition salts.

The term “drug resistant bacterium” as used herein is a bacterium which is able to survive exposure to at least one drug. In some embodiments the drug resistant bacterium is a bacterium which is able to survive exposure to a single drug or multiple drugs. Examples of drug resistant bacterium include but are not limited to vancomycin-resistant bacterium, methicilin-resistant bacterium, and β-lactam resistant bacterium.

An “implant” is any object intended for placement in a human body that is not a living tissue. Implants include naturally derived objects that have been processed so that their living tissues have been devitalized. As an example, bone grafts can be processed so that their living cells are removed, but so that their shape is retained to serve as a template for in growth of bone from a host. As another example, naturally occurring coral can be processed to yield hydroxyapatite preparations that can be applied to the body for certain orthopedic and dental therapies. An implant can also be an article comprising artificial components. The term “implant” can be applied to the entice spectrum of medical devices intended for placement in a human body.

“Medical device” refers to a non-naturally occurring object that is inserted or implanted in a subject or applied to a surface of a subject. Medical devices can be made of a variety of biocompatible materials, including: metals, ceramics, polymers, gels and fluids not normally found within the human body. Medical devices include scalpels, needles, scissors and other devices used in invasive surgical, therapeutic or diagnostic procedures; implantable medical devices, including artificial blood vessels, catheters and other devices for the removal or delivery of fluids to patients, artificial hearts, artificial kidneys, orthopedic pins, plates and implants; catheters and other tubes (including urological and biliary tubes, endotracheal tubes, peripherably insertable central venous catheters, dialysis catheters, long term tunneled central venous catheters peripheral venous catheters, short term central venous catheters, arterial catheters, pulmonary catheters, Swan-Ganz catheters, urinary catheters, peritoneal catheters), urinary devices (including long term urinary devices, tissue bonding urinary devices, artificial urinary sphincters, urinary dilators), shunts (including ventricular or arterio-venous shunts); prostheses (including breast implants, penile prostheses, vascular grafting prostheses, heart valves, artificial joints, artificial larynxes, otological implants), vascular catheter ports, wound drain tubes, hydrocephalus shunts, pacemakers and implantable defibrillators, and the like. Other examples will be readily apparent to practitioners in these arts.

Other surfaces related to health include the inner and outer aspects of those articles involved in water purification, water storage and water delivery, and those articles involved in food processing. Surfaces related to health can also include the inner and outer aspects of those household articles involved in providing for nutrition, sanitation or disease prevention. Examples can include food processing equipment for home use, materials for infant care, tampons and toilet bowls.

The polymer or polymeric nanocomposites coating can also be incorporated into glues, cements or adhesives, or in other materials used to fix structures within the body or to adhere implants to a body structure. Examples include polymethylmethacrylate and its related compounds, used for the affixation of orthopedic and dental prostheses within the body.

In one embodiment, compounds can be applied to or incorporated in certain medical devices that are intended to be left in position permanently to replace or restore vital functions such as ventriculoatrial, ventriculoperitoneal and dialysis shunts, and heart valves.

Other medical devices which can be coated include pacemakers and artificial implantable defibrillators, infusion pumps, vascular grafting prostheses, stents, suture materials, and surgical meshes.

Implantable devices intended to restore structural stability to body parts can be coated. Examples include implantable devices used to replace bones or joints or teeth.

Certain implantable devices are intended to restore or enhance body contours for cosmetic or reconstructive applications. Examples include breast implants, implants used for craniofacial surgical reconstruction and tissue expanders. Insertable devices include those objects made from synthetic materials applied to the body or partially inserted into the body through a natural or an artificial site of entry. Examples of articles applied to the body include contact lenses, stoma appliances, artificial larynx, endotracheal and tracheal tubes, gastrostomy tubes, biliary drainage tubes and catheters. Some examples of catheters that may be coated include peritoneal dialysis catheters, urological catheters, nephrostomy tubes and suprapubic tubes. Other catheter-like devices exist that may be coated include surgical drains, chest tubes and hemovacs.

Dressing materials and glues or adhesives used to stick the dressing to the skin may be coated.

As used herein, the term “microbicidal” means that the polymer or polymeric nanocomposites coating produces a substantial reduction in the amount of active microbes present on the surface, preferably at least one log kill, preferably at least two log kill, when an aqueous microbe suspension or an aerosol is applied at room temperature for a period of time, as demonstrated by the examples. In more preferred applications, there is at least a three log kill, most preferably a four log kill. Although 100% killing is typically desirable, it is generally not essential.

The present disclosure relates to a compound of Formula I

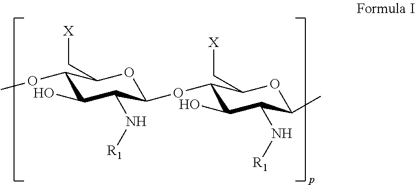

wherein:

### X is

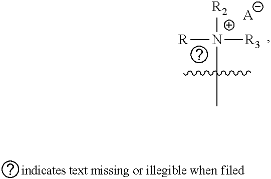

OH and combinations thereof;
R₂, R₃ and R₄ are independently selected from the group consisting of hydrogen, substituted or unsubstituted C₁₋₂₂ alkyl, substituted or unsubstituted C₆₋₁₀ aryl,

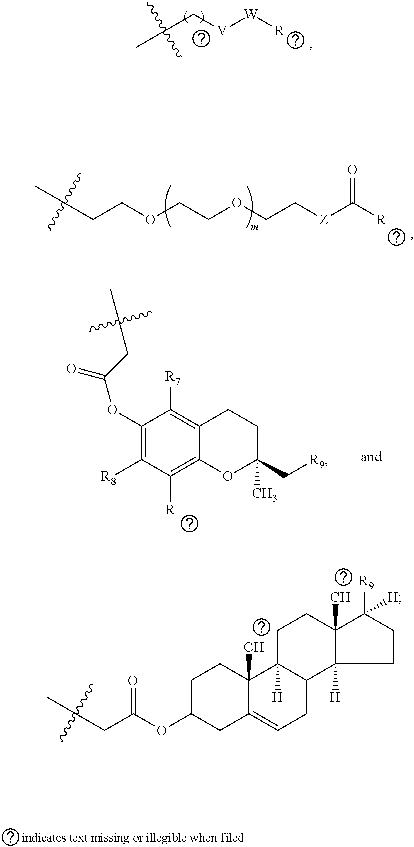

or
R₂ and R₃ taken together to form a substituted or unsubstituted cyclic ring system which is saturated or partially unsaturated and optionally have additional heteroatoms selected from O, N or S; or
R₂ and R₃ taken together to form a substituted or unsubstituted aromatic ring system optionally having heteroatoms selected from O, N or S; or
R₂, R₃ and R₄ may combine to form a substituted or unsubstituted bicylic ring system which is saturated, partially unsaturated or fully unsaturated, a substituted or unsubstituted aromatic ring system and optionally having heteroatoms selected from O, N or S;
V and W are independently selected from the group consisting of O, NH and —CO;

### Z is O or —NH;

R₁ is selected from the group consisting of hydrogen, C₁₋₁₆ alkyl, C₆₋₁₀ aryl, —COR₁₀, and combinations thereof;
R₅ and R₉ are independently selected from the group consisting of hydrogen, substituted or unsubstituted C₁₋₆ alkyl, substituted or unsubstituted C₂₋₂₄ alkenyl, substituted or unsubstituted C₆₋₁₀ aryl, and combinations thereof;
R₆, R₇ and R₈ are independently selected from hydrogen and methyl;
A^{⊖} is negatively charged counter anion;
R₁₀ is selected from the group consisting of C₁₋₁₆ alkyl and C₆₋₁₀ aryl, wherein alkyl and aryl are optionally substituted with halogen, alkyl, and aryl;
l is 0 to 4;
m is 0 to 3; and
p is 1 to 1000, wherein the degree of substitution of R₁ with hydrogen, C₁₋₁₆ alkyl, C₆₋₁₀ aryl, or —COR₁₀ in the compound of formula I is in the range of 20-100%; and the degree of substitution of X with

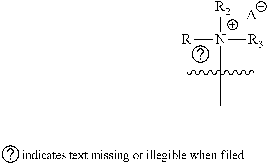

in the compound of formula I is in the range of 10-90%.

In one embodiment, the present disclosure relates to a compound of Formula I, wherein p is 2 to 1000.

In yet another embodiment, the present disclosure relates to a compound of Formula I, the degree of substitution of R₁ with C₁₋₆ alkyl, C₆₋₁₀ aryl, or —COR₁₀ in the compound of formula I is in the range of 20-100%; and the degree of substitution of X with

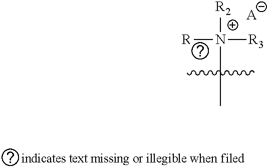

in the compound of formula I is in the range of 10-90%.

According to an embodiment, the present disclosure relates to a compound of Formula I

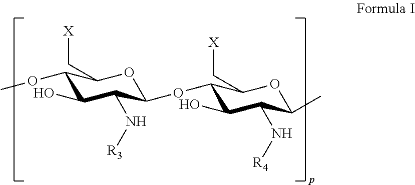

wherein:

### X is

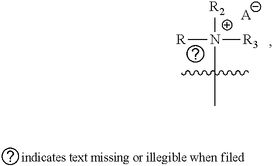

OH and combinations thereof;
R₂, R₃ and R₄ are independently selected from the group consisting of hydrogen, substituted or unsubstituted C₁₋₂₂ alkyl, substituted or unsubstituted C₆₋₁₀ aryl,

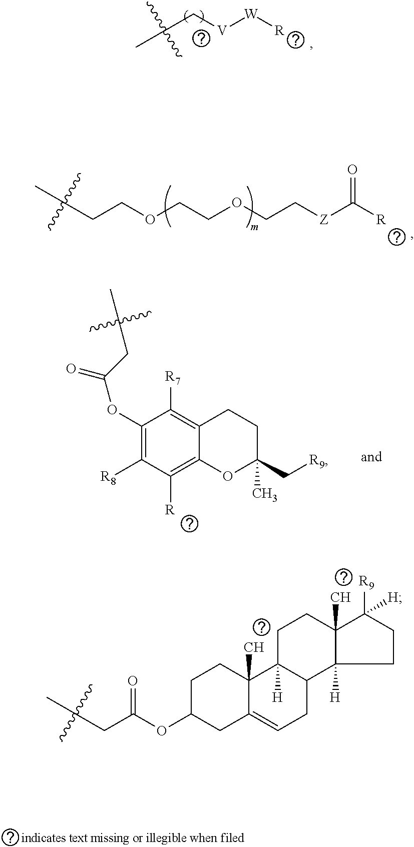

wherein alkyl, and aryl, are optionally substituted with one or more substituents selected from hydroxy, alkyl, aryl, alkoxy, halogen, haloalkyl, perhaloalkyl, cyano, OR₁₀, or
R₂ and R₃ taken together to form a substituted or unsubstituted cyclic ring system which is saturated or partially unsaturated and optionally having heteroatoms selected from O, N or S; or
R₂ and R₃ taken together to form a substituted or unsubstituted aromatic ring system optionally having heteroatoms selected from O, N or S and R₄ is absent; or
R₂, R₃ and R₄ may combine to form a substituted or unsubstituted bicylic ring system which is saturated, partially unsaturated or fully unsaturated, a substituted or unsubstituted aromatic ring system and optionally having heteroatoms selected from O, N or S; wherein the cyclic ring system, the aromatic ring system and the bicyclic ring system is further optionally substituted with 1 to 4 substituents independently selected from halo, alkyl, alkenyl, alkynyl, nitro, cyano, cycloalkyl, cycloalkylalkyl, aryl, arylalkyl, heterocyclyl, heterocyclylalkyl, heteroaryl, heteroarylalkyl and a compound of Formula II;

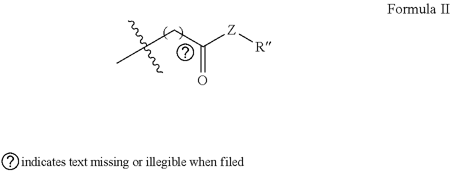

wherein the alkyl, aryl, heteroaryl is further optionally substituted with alkyl, cycloalkyl, cycloalkylalkyl, aryl, arylalkyl, heterocyclyl, heterocyclylalkyl, heteroaryl, heteroarylalkyl and a compound of Formula II,
V and W are independently selected from the group consisting of O, NH and —CO;

### Z is O or NH;

R″ is selected from the group consisting of C₁₋₂₂ alkyl, or C₂₋₂₄ alkenyl;
R₁ is-selected from the group consisting of hydrogen, C₁₋₁₆ alkyl; C₆₋₁₀ aryl, —COR₁₀ and combinations thereof;
R₅ and R₉ are independently selected from the group consisting of hydrogen, C₁₋₁₆ alkyl, C₂₋₂₄ alkenyl, C₆₋₁₀ aryl, and combinations thereof;
R₆, R₇ and R₈ are independently selected from hydrogen and methyl;
A^{⊖} is negatively charged counter anion;
R₁₀ is selected from the group consisting of C₁₋₆ alkyl and C₆₋₁₀ aryl, wherein alkyl and aryl are optionally substituted with halogen, alkyl, and aryl;
l is 0 to 4;
m is 0 to 3; and
p is 1 to 1000, wherein the degree of substitution of R₁ with hydrogen, C₁₋₁₆ alkyl, C₆₋₁₀ aryl, or —COR₁₀ in the compound of formula I is in the range of 20-100%; and the degree of substitution of X with

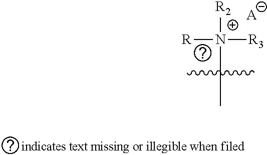

in the compound of Formula I is in the range of 10-90%.

According to another embodiment, the present disclosure relates to a compound of Formula I, wherein A^{⊖} is negatively charged counter anion selected from the group consisting of Cl⁻, Br⁻, I⁻, OH⁻, HCO₃⁻, CO₃²⁻, R¹¹COO⁻, R₁₁SO₄⁻, and R₁₁SO₃⁻, wherein R₁₁ is selected from the group consisting of hydrogen, C₁₋₆ alkyl and C₆₋₁₀ aryl, wherein alkyl and aryl are optionally substituted with hydroxyl, nitro, halogen, ester, alkyl, and aryl.

According to yet another embodiment, the present disclosure relates to a compound of Formula I, wherein X with

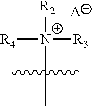

is selected from the group consisting of

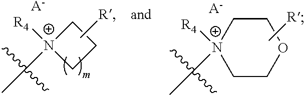

R₄ is selected from the group consisting of hydrogen, substituted or unsubstituted C₁₋₂₂ alkyl, substituted or unsubstituted C₆₋₁₀ aryl,

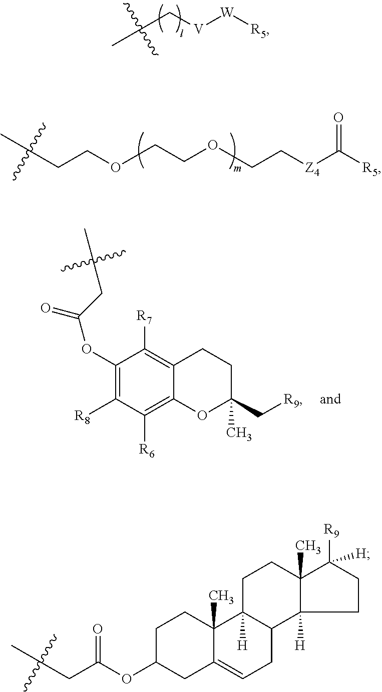

wherein alkyl, and aryl, are optionally substituted with one or more substituents selected from hydroxy, alkyl, aryl, alkoxy, halogen, haloalkyl, perhaloalkyl, cyano, —OR₁₀,
R′ is selected from the group consisting of alkyl, cycloalkyl, cycloalkylalkyl, aryl, arylalkyl, heterocyclyl, heterocyclylalkyl, heteroaryl, heteroarylalkyl and a compound of Formula II;

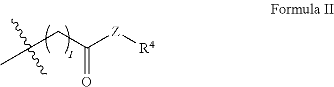

### Z is O or NH;

R″ is selected from the group consisting of C₁₋₂₂ alkyl, or C₂₋₂₄ alkenyl;
A^{⊖} is negatively charged counter anion selected from the group consisting of Cl⁻, Br⁻, I⁻, OH⁻, HCO₃⁻, CO₃²⁻, R₁₁COO⁻, R₁₁SO₄⁻, and R₁₁SO₃⁻;
V and W are independently selected from the group consisting of O, NH and —CO;
R₅ and R₉ are independently selected from the group consisting of hydrogen, C₁₋₁₆ alkyl, C₂₋₂₄ alkenyl, C₁₋₁₀ aryl, and combinations thereof;
R₆, R₇ and R₈ are independently selected from hydrogen and methyl;
R₁₀ is selected from the group consisting of C₁₋₁₆ alkyl and C₆₋₁₀ aryl, wherein alkyl and aryl are optionally substituted with halogen, alkyl, and aryl;
R₁₁ is selected from the group consisting of hydrogen, C₁₋₆ alkyl and C₆₋₁₀ aryl, wherein alkyl and aryl are optionally substituted with hydroxyl, nitro, halogen, ester, alkyl, and aryl;
l is 0 to 4; and
m is 0 to 3.

According to an embodiment, the resent disclosure relates to a compound of Formula I, wherein X with

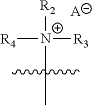

is selected from the group consisting of

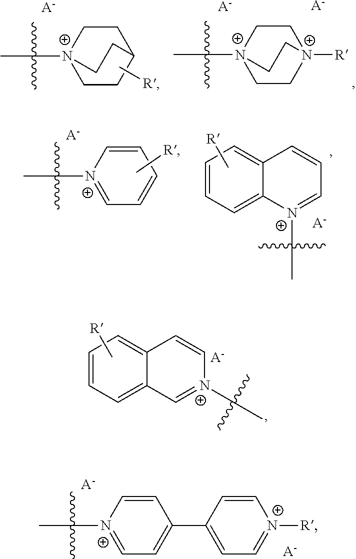

R′ is selected from the group consisting of alkyl, cycloalkyl, cycloalkylalkyl, aryl, arylalkyl, heterocyclyl, heterocyclylalkyl, heteroaryl, heteroarylalkyl and a compound of Formula II;

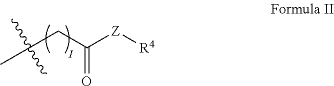

### Z is O or NH;

R″ is selected from the group consisting of C₁₋₂₂ alkyl, or C₂₋₂₄ alkenyl;
A^{⊖} is negatively charged counter anion selected from the group consisting of Cl⁻, Br⁻, I⁻, OH⁻, HCO₃⁻, CO₃²⁻, R₁₁COO⁻, R₁₁SO₄⁻, and R₁₁SO₃⁻;
R₁₁ is selected from the group consisting of hydrogen, C₁₋₆ alkyl and C₆₋₁₀ aryl, wherein alkyl and aryl are optionally substituted with hydroxyl, nitro, halogen, ester, alkyl, and aryl;
l is 0 to 4; and
m is 0 to 3.

According to another embodiment, the present disclosure relates to a compound of Formula I, wherein R₂, R₃, and R₄ are independently selected from the group consisting of

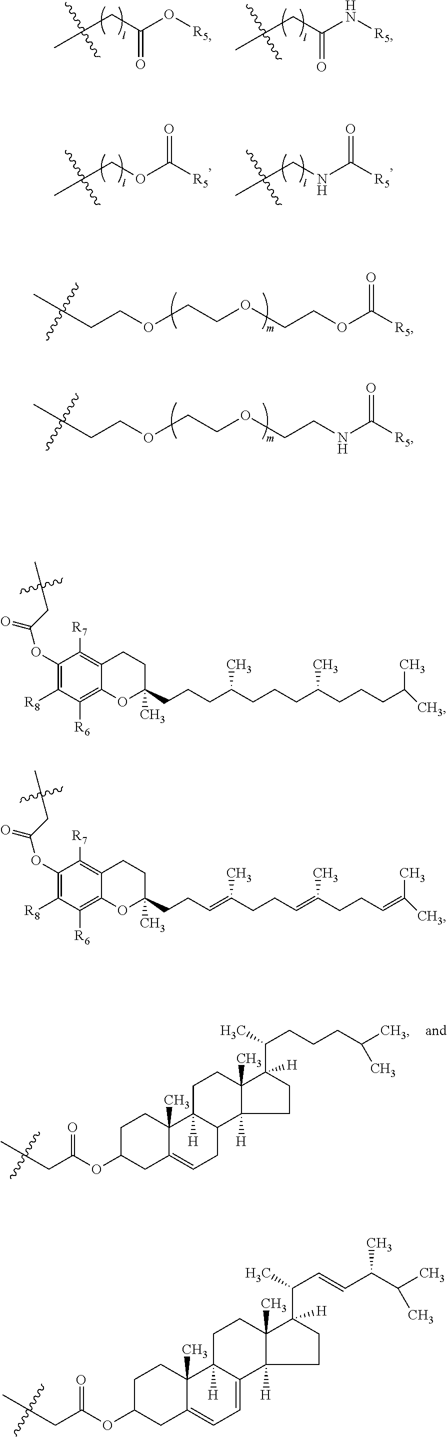

R₅ is selected from the group consisting of hydrogen, C₁₋₁₆ alkyl, C₂₋₂₄ alkenyl, C₆₋₁₀ aryl, and combinations thereof;

R₆, R₇ and R₈ are independently selected from hydrogen and methyl;
l is 0 to 4; and
m is 0 to 3.

According to yet another embodiment, the present disclosure relates to a compound of Formula I, wherein R₂ and R₃ are independently selected from the group consisting of hydrogen, C₁₋₂ alkyl;

R₄ is C₁₋₂₀ alkyl;
R₁ is independently selected from the group consisting of hydrogen, —COR₁₀, and combinations thereof;
A^{⊖} is selected from the group consisting of Cl⁻, Br⁻, R₁₁SO₃⁻;
R₁₀ is selected from the group consisting of C₁₋₁₆ alkyl and C₆₋₁₀ aryl, wherein alkyl and aryl are optionally substituted with halogen, alkyl, and aryl;
R₁₁ is selected from the group consisting of hydrogen, C₁₋₆ alkyl and C₆₋₁₀ aryl, wherein alkyl and aryl are optionally substituted with hydroxyl, nitro, halogen, ester, alkyl, and aryl, wherein the degree of substitution of R₁ with —COR₁₀ in the compound of formula I is in the range of 30-100%; and the degree of substitution of X with

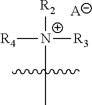

in the compound of formula I is in the range of 20-80%.

According to an embodiment, the present disclosure relates to a compound of Formula I, wherein R₂ and R₃ are independently methyl;

R₄ is C₁₂₋₁₆ alkyl;

### A^{⊖} is

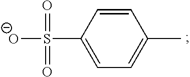

p is 500 to 900;
wherein the degree of substitution of X with

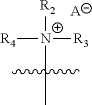

in the compound of formula I is in the range of 40-70%.

According to another embodiment, the present disclosure relates to a compound of Formula I, wherein:—

X is a combination of

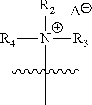

### and OH;

R₂ and R₃ is methyl;
R₁ is —COCH₃;
R₄ is C₁₂-C₁₆ alkyl;
p is an integer 700-800;

A^{⊖} is

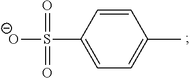

wherein the degree of substitution of X with

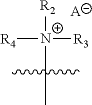

in the compound of formula I is in the range of 40-70%.

The present disclosure relates to the field of biotechnology and specifically to the development of polymeric antibacterial coatings. The present invention relates to the synthesis and characterization of water insoluble quaternized chitin derivatives designed to exhibit broad spectrum antibacterial activity, for example, against sensitive and/or multidrug-resistant Gram-positive and Gram-negative bacteria to be used as antibacterial coatings in medical devices and in house-hold applications.

The present disclosure relates to quaternized chitin derivatives which are completely insoluble in water and highly soluble in organic solvents, preferably selected from the group consisting of methanol, and DMSO. The compounds disclosed in the present disclosure are obtained from naturally occurring polymer chitin for development of antimicrobial coatings. They showed high antibacterial activity against various pathogens including drug resistant bacteria by disrupting the membrane integrity of the pathogens. These derivatives were almost equally active in mammalian fluids a primary requirement for the in-vivo applications. These compounds were highly selective towards bacteria over mammalian cell such hRBC and HEK 293 cell thus are hemocompatible and/or non-toxic. The compounds of the present disclosure were biodegraded in the presence of human enzyme lysozyme as the backbone of these derivatives, the naturally occurring polymer chitin, is susceptible towards lysozyme.

According to yet another embodiment, the present disclosure relates to a compound of Formula I for use in antimicrobial coatings. The organic solution of the compounds of formula I can be easily coated to prepare microbicidal paint. Biodegradable water insoluble antimicrobial paint to be used as antimicrobial coatings in various house-hold and bio-medical applications in order to prevent the bacterial infections especially nosocomial and medical device related infections.

The compounds disclosed in the present disclosure are soluble in aqueous solvents for use as antibacterial agents in the treatment of diseases caused by bacteria, fungi, and virus, preferably gram-positive and gram-negative bacteria.

The compounds disclosed in the present disclosure are insoluble in aqueous solvents and soluble in organic solvents thereof for use as antibacterial coatings in the prevention of diseases caused by bacteria, fungi, and virus, preferably gram-positive and gram-negative bacteria.

These compounds have a positive charge and a hydrophobic long chain/group can interact with the mostly negatively charged lipid membrane of the bacteria more strongly through improved electrostatic and van der Waal interactions. These increased interactions with bacterial cell membranes can serve as to kill the bacteria more efficiently. The compounds disclosed in the present disclosure can degrade in the presence of hydrolytic enzymes such lysozyme or chitinases suitable for the in-vivo as well as practical applications.

The coating disclosed in the present disclosure is done by spin coating, brush coating, dip coating or painting.

According to an embodiment, the present disclosure relates to a compound of Formula I for use as antibacterial agents in the treatment of diseases caused by bacteria, fungi, and virus.

According to another embodiment, the present disclosure relates to a compound of Formula I for use as antibacterial agents in the treatment of diseases caused by Gram-positive and Gram-negative bacteria.

The present disclosure relates to an article comprising a substrate, wherein the substrate is coated with or impregnated with the composition comprising the compound of Formula I, or the pharmaceutically acceptable salt.

An embodiment of the present disclosure relates to a pharmaceutical composition comprising a compound of Formula I with a pharmaceutically acceptable carrier, optionally in combination with one or more other pharmaceutical compositions.

The present disclosure further relates to a method of preparing biodegradable antimicrobial coatings and/or surfaces with or without pharmaceutical compositions.

The present disclosure relates to a bactericidal coating comprising a hydrophobic, water insoluble-polymer as disclosed in the present disclosure on an inert surface. The coating associates with the surfaces via non-covalent interactions.

The surface disclosed in the present disclosure is formed from material selected from the group consisting of metals, ceramics, glass, polymers, plastics, fibers and combinations thereof.

In an embodiment of the present disclosure the surface is the surface of a toy, bathroom fixture, countertop, tabletop, handle, computer, military gear, clothing, paper product, window, door, or interior wall fabric, gauze, tissue, surgical drape, air-filter, tubing, surgical instruments, device or implants to be placed into the body or tissue.

In some embodiments, the surface may be pretreated with an appropriate solution or suspension to modify the properties of the surface, and thereby strengthen the non-covalent interactions between the modified surface and the coating.

The polymer solution is applied to a surface at an appropriate temperature and for a sufficient period of time to form a coating on the surface, wherein the coating is effective in forming a microbicidal and optionally a bactericidal surface. Typical temperatures include room temperature, although higher temperatures may be used. Typical time periods include 20 minutes or less, 30 minutes or less, 60 minutes or less, and 120 minutes or less. In some embodiments the solution can be applied for 120 minutes or longer to form a coating with the desired antibacterial activity. However, preferably shorter time periods are used. The coatings are applied in an effective amount to form an antibacterial coating.

The present disclosure relates to a process of preparing a compound of Formula I, the process comprising:

(a) contacting a compound of Formula III, wherein R₁ and p are defined as above,

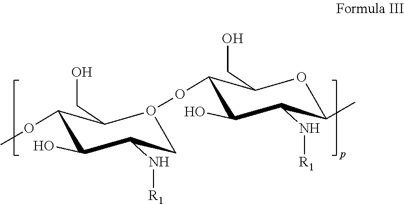

with R₁₁SO₃Cl, wherein R₁₁ is defined as above; in 0-10% wt/vol of lithium chloride and a solvent to obtain a compound of Formula IV, wherein R₁ and p are defined as above; Y is a combination of R₁₁SO₃— and OH, wherein the degree of substitution of Y with R₁₁SO₃— in the compound of Formula IV is in the range of 30-90%; with R₁₁SO₃— group at the C-6 position of Formula III.

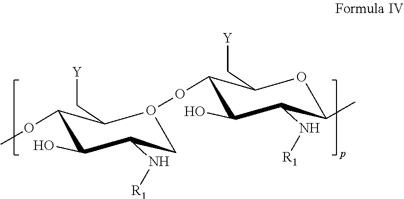

(b) reacting the compound of Formula IV with an acetylating agent in presence of a solvent to obtain an acetylated compound;
(c) treating the acetylated compound with a base to obtain O-deacetylated and N-acetalylated compound;
(d) contacting the O-deacetylated and N-acetalylated compound with NR₂R₃R₄, wherein R₂, R₃ and R₄ are defined as above, in presence of a solvent to obtain a solution; and
(e) cooling and precipitating the solution by a solvent to obtain a compound of Formula I wherein the degree of substitution of R₁ with hydrogen, C₁₋₁₆ alkyl, C₆₋₁₀ aryl, or —COR₁₀ in the compound of Formula I is in the range of 20-100%; and the degree of substitution of X with

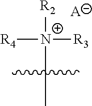

in the compound of Formula I is in the range of 10-90%.

The solvent disclosed in the present disclosure is selected from the group consisting of a polar solvent, non-polar solvent and mixtures thereof.

In an embodiment of the present disclosure, the polar solvent is selected from the group consisting of N,N-dimethylformamide, N,N-dimethylacetamide, N,N-dimethylsulfoxide, N-methyl-2-pyrrolidone, acetonitrile, acetone, chloroform, dichloromethane, 1,2-dichloroethane, methanol and mixtures, thereof, preferably N,N-dimethylacetamide and N,N-dimethylsulfoxide.

In another embodiment of the present disclosure, the non-polar solvent is selected from the group consisting of tetrahydrofuran, hexane, pentane, benzene and mixtures thereof.

In yet another embodiment of the present disclosure, the acetylating agent is selected from the group consisting of acetic anhydride, acetyl chloride, preferably acetic anhydride.

In an embodiment of the present disclosure, the base is selected from the group consisting of potassium hydroxide, sodium hydroxide, barium hydroxide, cesium hydroxide, strontium hydroxide, calcium hydroxide, lithium hydroxide, and rubidium hydroxide preferably potassium hydroxide.

An embodiment of the present disclosure relates to a process of preparing a compound of Formula I, the process comprising:

(a) contacting a compound of Formula III, wherein R₁ is independently selected from the group consisting of hydrogen, —COR₁₀, and combinations thereof; R₁₀ is C_{1 a}lkyl; and p is 700 to 800,

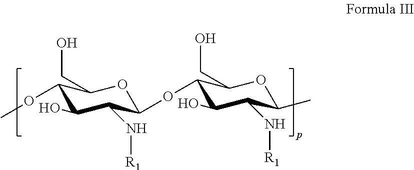

with R₁₁SO₃Cl, wherein R₁₁ is C₆ aryl, wherein aryl is substituted with alkyl; in 5% wt/vol of lithium chloride-N,N-dimethylacetamide solvent system to obtain a compound of formula IV with R₁₁SO₃— group at the C-6 position of formula III.
wherein R₁ is independently selected from the group consisting of hydrogen, —COR₁₀, and combinations thereof; R₁₀ is C₁ alkyl; Y is a combination of R₁₁SO₃— and OH, and p is 700 to 800.
wherein the degree of substitution of Y with R₁₁SO₃— in the compound of Formula IV is in the range of 30-90%;

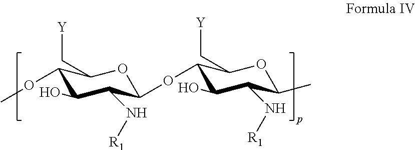

(b) reacting the compound of Formula IV with an acetic anhydride in presence of methanol to obtain an acetylated compound;
(c) treating the acetylated compound with a methanolic potassium hydroxide to obtain O-deacetylated and N-acetalylated compound;
(d) contacting the O-deacetylated and N-acetalylated compound with NR₂R₃R₄ selected from the group consisting of N,N-dimethyl dodecylamine, N,N-dimethyl tetradecylamine or N,N-dimethyl hexadecylamine in presence of a solvent selected from N,N-dimethyl acetamide or N,N-dimethyl sulfoxide to obtain a solution;
(e) cooling and precipitating the solution by a solvent selected from the group consisting of diethylether, n-hexane, acetone and combinations thereof to obtain a compound of Formula I.
wherein the degree of substitution of R₁ with hydrogen, C₁₋₆ alkyl, C₆₋₁₀ aryl, or —COR₁₀ in the compound of formula I is in the range of 30-100%; and the degree of substitution of X with

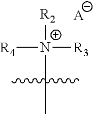

in the compound of Formula I is in the range of 40-70%.

The tosylation of the polymer chitin in DMAc/LiCl (5% wt/vol) solvent system could not be achieved without triethylamine. The most likely reason is to the generation of hydrogen chloride (HCl) during tosylation. It is known that the pyranose units of chitin connected by ether bonds are vulnerable to acid. Therefore, tosylation in the absence of triethylamine generates an acid environment that leads to degradation of chitin. The presence of triethylamine acts to neutralize the HCl generated by tosylation. The reaction temperature, reaction time, and the ratio of tosyl chloride to chitin were the three main parameters that influence the homogeneous C-6 tosylation of chitin. Two reasons favor tosylation under low-temperature conditions. First, low temperatures support the control of regioselectivity by the tosyl group better. It is well-known that the C-6 hydroxyl of chitin is more reactive than the C-3 hydroxyl as it is sterically less hindered, an effect that becomes ineffective as the temperature is increased as both hydroxyls become equally reactive. Second, although the chloro anion is not a strong nucleophile, chlorination may still be induced at higher temperature in the presence of the LiCl-DMAc binary solvent system. To avoid these two disadvantages, all tosylation was carried out at low temperature (8° C.).

The present disclosure relates to a process of making nanocomposites by using compounds of Formula I, the process comprising: (a) dissolving a compound of Formula I in an organic solvent; (b) adding to a solution of silver slat of formula R-M in another organic solvent; and (c) keeping the mixture at room temperature for 6-72 h.

In an embodiment of the present disclosure, the R is selected from the group consisting of NO₃⁻, Cl⁻, R′COO⁻, R′SO₃⁻, R′SO₂N—; wherein R′ is selected from the group consisting of C₁₋₁₆ acyclic or cyclic alkyl and C₆₋₁₀ aryl, wherein alkyl and aryl are optionally substituted with halogen, alkyl, and aryl; M is selected from the group of silver, or gold, preferably silver.

In another embodiment of the present disclosure, the organic solvents are selected from the group consisting of N,N-dimethylformamide, N,N-dimethylacetamide, dimethylsulfoxide, N-methyl-2-pyrrolidone, acetone, methanol, ethanol, water and combinations thereof, preferably selected from the group of methanol, dimethylsulfoxide, and combinations thereof, more preferably methanol and dimethyl sulfoxide.

The present disclosure relates to an antibacterial polymeric nanocomposite. The present disclosure further relates to a process for the preparation of antibacterial polymeric nanocomposites. Different type of nanoparticles, for example silver nanoparticles and gold nanoparticles can be prepared by the method described in the present disclosure. The antibacterial polymeric nanocomposites were prepared in-situ by adding solution of silver para-toluene sulfonate (AgPTS) in DMSO in to a solution of chitin derivatives of formula I in methanol at a ratio 1:1 and 0.5:1 (wt/wt) and keeping the mixture at room temperature for about 48 h. In-situ formation of silver nanoparticles (Ag NPs) was observed within 6 h where the chitin derivatives and/or DMSO act as reducing agents and chitin derivatives as stabilizing agent. As the method of formation is independent of the degree of quaternization and type of quaternary ammonium groups, any polymer from the Formula I of the current patent could be used to synthesize silver nanoparticle in-situ.

The present disclosure relates to a nanocomposite for use as antibacterial agents in the treatment of diseases caused by bacteria, fungi, and virus.

The nanocomposite as disclosed in the present application is use in antimicrobial coatings.

The present disclosure relates to an article comprising a substrate, wherein the substrate is coated with or impregnated with the composition comprising the nanocomposite, or the pharmaceutically acceptable salt.

The present disclosure further relates to a pharmaceutical composition comprising a nanocomposite with a pharmaceutically acceptable carrier, optionally in combination with one or more other pharmaceutical compositions.

The synthesized compounds disclosed in the present disclosure are characterized by FT-IR, ¹HNMR, ¹³CNMR and elemental analysis.

The formation of silver nanoparticle was confirmed by UV-visible absorption spectroscopy and transmission electron microscopy (TEM). UV-visible absorption spectroscopy showed the appearance of surface plasmon band for silver nanoparticle at 410 nm. TEM images showed that size of these nanoparticles ranges from 20-200 nm. It was found that these composite system not only highly antibacterial but also act synergistically against both Gram-positive as well as Gram-negative bacteria. Specially, these composites were found to be highly active against Gram-negative bacteria which are very difficult to treat. These composites would be acting by both release and contact based mechanisms and would show long-lasting action. Further, as the required for antibacterial activity is very less, the toxicity towards mammalian cells would be reduced even further.

Embodiments can be compatible for combination with currently employed antiseptic regimens to enhance their antimicrobial efficacy or cost-effective use. Selection of an appropriate vehicle for bearing a compound will be determined by the characteristics of the particular use.

The prepared nanocomposites were characterized by UV-visible and transmission electron microscopy (TEM).

### Abbreviations

The following abbreviations are employed in the examples and elsewhere herein:

LiCl: Lithium chloride,
HCl: Hydrochloric acid,
KOH: Potassium hydroxide,
KBr: Potassium bromide,

### NEt₃: Triethylamine,

TsCl: p-Toluenesulfonyl chloride,

### H₂O: Water,

### CHCl₃: Chloroform,

CH₂Cl₂: Dichloromethane,

### MeOH: Methanol,

### EtOH: Ethanol,

n-BuOH: n-Butanol,

### DMF: N,N-Dimethylformamide,

### DMAc: N,N-Dimethylacetamide,

### DMSO: N,N-Dimethylsulfoxide,

### THF: Tetrahydrofuran,

### PMMA: Poly(methylmethacrylate),

PLA: Poly(lactic acid),
PLGA: Poly(lactic-co-glycolic) acid,
o: ortho,
m: meta,
s: singlet,
d: doublet,
t: triplet,
m: multiplet,
MIC: Minimum inhibitory concentration,
MBC: Minimum bactericidal concentration,
CFU: Colony forming unit,

### LB: Luria-Bertani,

PBS: Phosphate buffer saline,
TSB: Tryptic soy broth,
hRBC: Human red blood cell,

### HEK: Human Embryonic Kidney,

OFN: oxygen-free nitrogen,
DA: Degree of acetylation,
DS: Degree of substitution,
DQ: Degree of quaternization,
MRSA: Methicillin-resistant S. aureus,
VRE: Vancomycin-resistant enterococci,

### FT-IR: Fourier Transform Infrared Spectroscopy,

### NMR: Nuclear Magnetic Resonance Spectroscopy,

### UV: Ultra-violet,

### FESEM: Field Emission Scanning Electron Microscopy,

TEM: Transmission electron microscopy.

The compounds of Formula I may be prepared as outlined in the Scheme 1 below:

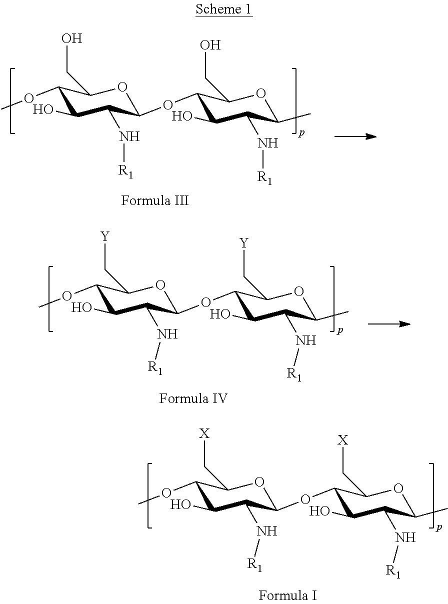

### EXAMPLES

The disclosure is further illustrated by the following examples which in no way should be construed as being further limiting. One skilled in the art will readily appreciate that the specific methods and results described are merely illustrative.

Chitin with a degree of acetylation (DA) ˜75% and potassium hydroxide (KOH) were purchased from SD Fine, India. Lithium chloride, triethylamine (NEt₃), acetic anhydride, p-toluenesulfonyl chloride, silver para-toluene sulfonate, N,N-dimethyltetradecylamine, N,N-dimethylhexadecylamine were obtained from Sigma-Aldrich, USA. N,N-dimethyldodecylamine was purchased from Across Organics, Belgium. Anhydrous N,N dimethylacetamide (DMAc) was obtained from Sigma-Aldrich, USA. All other solvents were purchased from SD Fine, India and were off analytical grade. Methanol was dried with calcium hydride and stored over 4 Å molecular sieves. Triethylamine was dried with KOH and stored over KOH. Bacterial strains, S. aureus MTCC 737, E. coli MTCC 447 and P. aeruginosa (MTCC 424) were purchased from MTCC (Chandigarh, India). Vancomycin-resistant enterococci (VRE), β-lactam-resistant K. pneumoniae (ATCC 700603), Methicillin-resistant S. aureus (MRSA) (ATCC 33591) were obtained from ATCC (Rockville, Md.). Human RBCs were used for hemolytic assay and Human Embryonic Kidney 293 (HEK 293) cells were used for cytotoxicity assay. Nuclear magnetic resonance spectra (NMR) were recorded on a Bruker AMX-400 instrument (400 MHz) in deuterated solvents. FT-IR spectra of the solid compounds were recorded on Bruker IFS66 V/s spectrometer using KBr pellets. Elemental analysis was performed in a Thermo Finnigan FLASH EA 1112 CHNS analyzer. UV-Visible spectra were taken by Perkin Elmer Lambda 900 UV/Vis/NIR spectrometer. TEM was performed on a Technai F30 UHR version electron microscope, using a field emission gun (FEG) operating at an accelerating voltage of 200 kV. Fluorescence microscopy images were captured with a Leica DM 2500 fluorescence microscope. A WS5000 spin coater, Techno India, India was used for making polymer coatings. Eppendorf 5810R centrifuge was used. TECAN (Infinite series, M200 pro) Plate Reader was used to measure absorbance and fluorescence.

### Example 1: Preparation of Tosylchitin 1

A 2.0 g amount of chitin (equivalent to ˜10 mmol of pyranose unit) and 5.2 g of lithium chloride (LiCl) dried respectively at 80° C. overnight and at 130° C. for 4 h and cooled, were both placed in a 250 mL three necked flask fitted with rubber septa. The flask was purged with dry oxygen-free nitrogen (OFN), and 104 mL of anhydrous DMAc was added via a syringe. The mixture was stirred at room temperature using a magnetic stirrer bar until all solids were dissolved. To the resultant chitin solution was added Et₃N (28.8 mL, 208 mmol) and the reaction flask was transferred to a cold incubator at 8° C. and the mixture was cooled. A solution of tosyl chloride (38.12 g, 200 mmol) in 48 mL of DMAc was then added to the reaction mixture and the reaction was allowed to proceed at 8° C. for 24 h with stirring. At the end of the reaction, the mixture was filtered to remove the insoluble solids and to the filtrate 500 mL of acetone was added to precipitate the product. The precipitate was washed with methanol (100 mL×4), 400 mL of water (100 mL×4), and 400 mL of acetone (100 mL×4) to obtain white or yellowish white tosylchitin 1.

The degree of substitution (DS) was determined by the S/N ratio of elemental analysis. For the ratio of 20:1 for TsCl to pyranose, the reaction time of 24 h gives tosylchitin with DS of 0.65-0.75 and yield of around 80%.

### Example 2: Preparation of Tosylchitin 2

A 2.0 g amount of chitin (equivalent to ˜10 mmol of pyranose unit) and 5.2 g of lithium chloride (LiCl) dried respectively at 80° C. overnight and at 130° C. for 4 h and cooled, were both placed in a 250 mL three necked flask fitted with rubber septa. The flask was purged with dry oxygen-free nitrogen (OFN), and 104 mL of anhydrous DMAc was added via a syringe. The mixture was stirred at room temperature using a magnetic stirrer bar until all solids were dissolved. To the resultant chitin solution was added Et₃N (28.8 mL, 208 mmol) and the reaction flask was transferred to a cold incubator at 8° C. and the mixture was cooled. A solution of tosyl chloride (38.12 g, 200 mmol) in 48 mL of DMAc was then added to the reaction mixture and the reaction was allowed to proceed at 8° C. for 48 h with stirring. At the end of the reaction, the mixture was filtered to remove the insoluble solids and to the filtrate 500 mL of acetone was added to precipitate the product. The precipitate was washed with methanol (100 mL×5), 400 mL of water (100 mL×4), and 400 mL of acetone (100 mL×4) to obtain white or yellowish white tosylchitin 2.

The degree of substitution (DS) was determined by the S/N ratio of elemental analysis. For the ratio of 20:1 for TsCl to pyranose, the reaction time of 48 h gives tosylchitin with DS of 0.70-0.75 and yield of around 80%.

### Example 3: Preparation of Tosylchitin 3

A 2.0 g amount of chitin (equivalent to ˜10 mmol of pyranose unit) and 5.2 g of lithium chloride (LiCl) dried respectively at 80° C. overnight and at 130° C. for 4 h and cooled, were both placed in a 250 mL three necked flask fitted with rubber septa. The flask was purged with dry oxygen-free nitrogen (OFN), and 104 mL of anhydrous DMAc was added via a syringe. The mixture was stirred at room temperature using a magnetic stirrer bar until all solids were dissolved. To the resultant chitin solution was added Et₃N (28.8 mL, 208 mmol) and the reaction flask was transferred to a cold incubator at 8° C. and the mixture was cooled. A solution of tosyl chloride (38.12 g, 200 mmol) in 48 mL of DMAc was then added to the reaction mixture and the reaction was allowed to proceed at 8° C. for 72 h with stirring. At the end of the reaction, the mixture was filtered to remove the insoluble solids and to the filtrate 500 mL of acetone was added to precipitate the product. The precipitate was washed with methanol (100 mL×6), 400 mL of water (100 mL×4), and 400 mL of acetone (100 mL×4) to obtain white or yellowish white tosylchitin 3.

The degree of substitution (DS) was determined by the S/N ratio of elemental analysis. For the ratio of 20:1 for TsCl to pyranose, a reaction time of 72 h gave tosylchitin with DS of 0.80-0.90 and yield of around 80%.

Three different types of tosylchitin were prepared (Table 1).

TABLE 1
Synthetic conditions and yield of tosylchitins

| | TsCl | Reaction | Reaction | |
|---|---|---|---|---|
| | used/sugar unit | time | temperature | |
| Sample | (equivalent) | (h) | (° C.) | % of Yield |
| Tosylchitin 1 | 20 | 24 | 8 | ~80 |
| Tosylchitin 2 | 20 | 48 | 8 | ~80 |
| Tosylchitin 3 | 20 | 72 | 8 | ~80 |

### Example 4: N-acetylation of Tosylchitin 1

A 2.55 g amount of tosylchitin 1, prepared in example 1 was suspended in anhydrous methanol (55 mL). Acetic anhydride (820 μL) was added to the methanolic suspension of tosylchitin 1 and the reaction was allowed to proceed overnight. After the reaction, the acetylated tosylchitin was filtered and washed with methanol and diethylether repeatedly. Finally, acetylated tosyl-chitin was treated with 0.1% methanolic potassium hydroxide (65 mL) for 3 h to give N-acetylated tosyl-chitin 1.

FT-IR (KBr): υ 3400 cm⁻¹ (OH str.), 1660 (Amide I, C═O str.), 1600 (phenylene), 1550 (Amide II, C═O str.), 1175 (SO₂), and 815 (phenylene); ¹HNMR (DMSO-d₆, 400 MHz): δ 1.893 (s, —NHCOCH₃), 2.285 (s, —O₂S—C₆H₄—CH₃), 3.724-4.861 (m, Cell-H), 7.120 (d, SO₃—C₆H₄—CH₃, m-H); 7.474 (d, SO₃—C₆H₄—CH₃, o-H), 7.787 (broad, —NHCOCH₃); ¹³CNMR (DMSO-d₆, 400 MHz): δ 21.5, 23.1, 54.7, 68.0-72.7, 79.7, 100.5, 125.4, 128.2, 130.5, 137.8, 169.5; Elemental analysis: C: 48.98, H: 5.82, N: 5.29, S: 4.84 (Calculated); C: 48.32, H: 5.84, N: 5.32, S: 4.89 (Found).

### Example 5: N-acetylation of Tosylchitin 2

A 2.55 g amount of tosylchitin 2, prepared in example 2 was suspended in anhydrous methanol (60 mL). Acetic anhydride (760 μL) was added to the methanolic suspension of tosylchitin 2 and the reaction was allowed to proceed overnight. After the reaction, the acetylated tosylchitin was filtered and washed with methanol and diethylether repeatedly. Finally, the acetylated tosyl-chitin was treated with 0.1% methanolic potassium hydroxide (60 mL) for 3 h to give N-acetylated tosyl-chitin 2.

FT-IR (KBr): υ 3400 cm⁻¹ (OH str.), 1665 (Amide I, C═O str.), 1600 (phenylene), 1555 (Amide II, C═O str.), 1175 (SO₂), and 815 (phenylene); ¹HNMR (DMSO-d₆, 400 MHz): δ 1.894 (s, —NHCOCH₃), 2.285 (s, —O₂S—C₆H₄—CH₃), 3.764-4.852 (m, Cell-H), 7.112 (d, SO₃—C₆H₄—CH₃, m-H), 7.452 (d, SO₃—C₆H₄—CH₃, o-H), 7.796 (broad, —NHCOCH₃); ¹³CNMR (DMSO-d₆, 400 MHz): δ 21.3, 23.5, 54.4, 68.2-72.7, 79.5, 100.5, 125.1, 128.7, 130.6, 137.8, 169.7; Elemental analysis: C: 49.28, H: 5.71, N: 5.0, S: 5.71 (Calculated); C: 48.74, H: 5.96, N: 5.1, S: 5.77 (Found).

### Example 6: N-acetylation of Tosylchitin 3

A 2.55 g amount of tosylchitin 3, prepared in example 3 was suspended in anhydrous methanol (60 mL). Acetic anhydride (710 μL) was added to the methanolic suspension of tosylchitin 3 and the reaction was allowed to proceed overnight. After the reaction, the acetylated tosylchitin was filtered and washed with methanol and diethylether repeatedly. Finally, the acetylated tosyl-chitin was treated with 0.1% methanolic potassium hydroxide (55 mL) for 3 h to give N-acetylated tosylchitin 3.

FT-IR (KBr): υ 3450 cm⁻¹ (OH str.), 1660 (Amide I, C═O str.), 1600 (phenylene), 1550 (Amide II, C═O str.), 1175 (SO₂), and 815 (phenylene); ¹HNMR (DMSO-d₆, 400 MHz): δ 1.893 (s, —NHCOCH₃), 2.286 (s, —O₂S—CH₄—CH₃), 3.728-4.825 (m, Cell-H), 7.112 (d, SO₃—C₆14-CH₃, m-H), 7.472 (d, SO₃—C₆H₄—CH₃, o-H), 7.778 (broad, —NHCOCH₃); 3CNMR (DMSO-d₆, 400 MHz): δ 21.7, 23.9, 55.1, 67.8-72.9, 78.9, 101.1, 125.7, 128.4, 130.6, 137.2, 169.1; Elemental analysis: C: 49.56, H: 5.62, N: 4.74, S: 6.5 (Calculated); C: 49.31, H: 5.87, N: 4.81, S: 6.42 (Found).

The degree of acetylation (DA) of purified chitin was about 75%. The presence of 25% free amino groups may influence, undesirably, the subsequent tosylchitin neucleophilic substitution (S_{N}2) reactions as well as add complexity, when estimating the degree of substitution in the chitin derivatives by elemental analyses. The conditions for the homogeneous tosylation of chitin are mild and deacetylation unlikely. Therefore, if 100% acetylated chitin was utilized, the N-acetylation of tosylchitin, which is tedious, could be avoided. The poor solubility and swellability of chitin in common organic solvents is the most likely cause for the disappointing results of N-acetylation in methanol, while concurrent 6-O-acetylation complicated the reaction in DMAc/LiCl. This method of preparing tosylchitin and subsequent acetylation was satisfactory overall, but some O-acetylation was observed at 1750 cm⁻¹ in the FT-IR spectrum of N-acetylated tosylchitin. A further alkali treatment step was therefore imposed to give N-acetylated tosylchitin. It is noted that compared to chitin, tosylchitin has good swellability in methanol that made the N-acetylation of tosylchitin in methanol much more efficient. However, it was observed that while O-deacetylation, detosylation of the tosylchitin occurred to some extent (Table 2).

TABLE 2
Properties of tosyl chitins

| | DS before N- | DS after N- | |
|---|---|---|---|
| Sample | acetylation^{a} | acetylation^{a} | Yield (%) |
| tosylchitin 1 | 65 | ~40 | ~80 |
| tosylchitin 2 | 75 | ~50 | ~80 |
| tosylchitin 3 | 85 | ~60 | ~80 |

^{a}DS = degree of substitution

### Example 7: Preparation of 1a

Tosylchitin 1 (1.0 g) with degree of tosylation ˜40% was first suspended in anhydrous N,N-dimethylacetamide (DMAc) (30 mL) in sealed screw-top pressure tube. To the suspension N,N-dimethyldodecyamine (5.2 mL) was added and the reaction mixture was stirred at 120° C. for 96 h. After the reaction is over, diethyl ether was added in excess (150 mL) to precipitate the product. The precipitate was then filtered through a sintered glass funnel and was washed repeatedly with diethyl ether. White colored compounds with 100% degree of quaternization with respect to tosylated group was obtained (75-80% yield). FT-IR: υ 3415 cm⁻¹ (OH str.), 2925 cm⁻¹ (—CH₂— assym. str.), 2850 cm⁻¹ (—CH₂— sym. str.), 1680 cm⁻¹ (Amide I, C═O str.), 1630 cm⁻¹ (phenylene), 1560 cm⁻¹ (Amide II, NH ben.), 1470 cm⁻¹ (—CH₂— scissor), 1380 cm⁻¹ (SO₂, asymmetric); 1170 cm⁻¹ (SO₂, symmetric); ¹HNMR: (DMSO-d₆, 400 MHz): δ 0.845 (t, —CH₃(CH₂)₁₁—N⁺(CH₃)₂—, 3H), 1.252 (m, —CH₃(CH₂)₉CH₂CH₂—N⁺(CH₃)₂—, 18H), 1.696 (m, —CH₃(CH₂)₉CH₂CH₂—N⁺(CH₃)₂—, 2H), 1.893 (s, —NHCOCH₃), 2.281 (s, SO₃—C₆H₄—CH₃), 3.063-4.866 (m, Cell-H and —CH₃(CH₂)₉CH₂CH₂—N⁺(CH₃)₂—), 7.093 (d, SO₃—C₆H₄—CH₃, m-H), 7.514 (d, SO₃—C₆H₄—CH₃, o-H); Elemental analysis: C: 56.11, H: 7.94, N: 5.60, S: 3.66 (Calculated); C: 55.81, H: 7.98, N: 5.42, S: 3.50 (Found).

### Example 8: Preparation of 1b

Tosylchitin 1 (1.0 g) with degree of tosylation ˜40% was first suspended in anhydrous N,N-dimethylacetamide (DMAc) (30 mL) in sealed screw-top pressure tube. To the suspension N,N-dimethyltetradecyamine (6.2 mL) was added and the reaction mixture was stirred at 120° C. for 96 h. After the reaction is over, diethyl ether was added in excess (150 mL) to precipitate the product. The precipitate was then filtered through a sintered glass funnel and was washed repeatedly with diethyl ether. White colored compounds with 100% degree of quaternization with respect to tosylated group was obtained (75-80% yield). FT-IR: υ 3415 cm⁻¹ (OH str.), 2930 cm⁻¹ (—CH₂— assym. str.), 2855 cm⁻¹ (—CH₂— sym. str.), 1680 cm⁻¹ (Amide I, C═O str.), 1630 cm⁻¹ (phenylene), 1560 cm⁻¹ (Amide II, NH ben.), 1470 cm⁻¹ (—CH₂— scissor), 1380 cm⁻¹ (SO₂, asymmetric), 1170 cm⁻¹ (SO₂, symmetric); ¹HNMR: (DMSO-d₆, 400 MHz): δ 0.890 (t, —CH₃(CH₂)₁₁—N⁺(CH₃)₂—, 3H), 1.254 (m, —CH₃(CH₂)₉CH₂CH₂—N⁺(CH₃)₂—, 18H), 1.698 (m, —CH₃(CH₂)₉CH₂CH₂—N⁺(CH₃)₂—, 2H), 1.893 (s, —NHCOCH₃), 2.281 (s, SO₃—C₆H₄—CH₃), 3.063-4.866 (m, Cell-H and —CH₃(CH₂)₉CH₂CH₂—N⁺(CH₃)₂—), 7.102 (d, SO₃—C₆H₄—CH₃, m-H), 7.502 (d, SO₃—C₆H₄—CH₃, o-H); Elemental analysis: C: 57.17, H: 8.14, N: 5.43, S: 3.55 (Calculated); C: 56.90, H: 8.29, N: 5.26, S: 3.50 (Found).

### Example 9: Preparation of 1c

Tosylchitin 1 (1.0 g) with degree of tosylation ˜40% was first suspended in anhydrous N,N-dimethylacetamide (DMAc) (30 mL) in sealed screw-top pressure tube. To the suspension N,N-dimethylhexadecyamine (6.8 mL) was added and the reaction mixture was stirred at 120° C. for 96 h. After the reaction is over, diethyl ether was added in excess (150 mL) to precipitate the product. The precipitate was then filtered through a sintered glass funnel and was washed repeatedly with diethyl ether. White colored compounds with 100% degree of quaternization with respect to tosylated group was obtained (75-80% yield). FT-IR: υ 3420 cm⁻¹ (OH str.), 2920 cm⁻¹ (—CH₂— assym. str.), 2850 cm⁻¹ (—CH₂— sym. str.), 1675 cm⁻¹ (Amide I, C═O str.), 1630 cm⁻¹ (phenylene), 1565 cm⁻¹ (Amide II, NH ben.), 1470 cm⁻¹ (—CH₂— scissor), 1375 cm⁻¹ (SO₂, asymmetric), 1165 cm⁻¹ (SO₂, symmetric); ¹HNMR: (DMSO-d₆, 400 MHz): δ 0.870 (t, —CH₃(CH₂)₁₁—N⁺(CH₃)₂—, 3H), 1.248 (m, —CH₃(CH₂)₉CH₂CH₂—N⁺(CH₃)₂—, 18H), 1.598 (m, —CH₃(CH₂)₉CH₂CH₂—N⁺(CH₃)₂—, 2H), 1.798 (s, —NHCOCH₃), 2.232 (s, SO₃—C₆H₄—CH₃), 3.103-4.956 (m, Cell-H and —CH₃(CH₂)₉CH₂CH₂—N⁺(CH₃)₂—), 7.120 (d, SO₃—C₆H₄—CH₃, m-H), 7.542 (d, SO₃—C₆H₄—CH₃, o-H); Elemental analysis: C: 58.03, H: 8.32, N: 5.27, S: 3.44 (Calculated); C: 57.90, H: 8.54, N: 5.16, S: 3.28 (Found).

### Example 10: Preparation of 2a

Tosylchitin 2 (1 g) with degree of tosylation ˜50% was first dissolved in N,N-dimethylacetamide (DMAc) (35 mL) in sealed screw-top pressure tube. To the solution N,N-dimethyldodecyamine (6.8 mL) was added and the reaction mixture was heated at 120° C. for 96 h. After the reaction is over, diethyl ether was added in excess (150 mL) to precipitate the product, filtered through a sintered glass funnel and was washed repeatedly with diethyl ether to give white colored compound with 100% degree of quaternization with respect to tosylated group (75-80% yield). FT-IR: υ 3415 cm⁻¹ (OH str.), 2925 cm⁻¹ (—CH₂— assym. str.), 2850 cm⁻¹ (—CH₂— sym. str.), 1680 cm⁻¹ (Amide I, C═O str.), 1630 cm⁻¹ (phenylene), 1560 cm⁻¹ (Amide II, NH ben.), 1470 cm⁻¹ (—CH₂-scissor), 1380 cm⁻¹ (SO₂, asymmetric), 1170 cm⁻¹ (SO₂, symmetric); ¹HNMR: (DMSO-d₆, 400 MHz): δ 0.872 (t, —CH₃(CH₂)₁₁—N⁺(CH₃)₂—, 3H), 1.242 (m, —CH₃(CH₂)₉CH₂CH₂—N⁺(CH₃)₂—, 18H), 1.686 (m, —CH₃(CH₂)₉CH₂CH₂—N⁺(CH₃)₂—, 2H), 1.883 (s, —NHCOCH₃), 2.281 (s, SO₃—C₆H₄—CH₃), 3.023-4.966 (m, Cell-H and —CH₃(CH₂)₉CH₂CH₂—N⁺(CH₃)₂—), 7.123 (d, SO₃—C₆H₄—CH₃, m-H), 7.524 (d, SO₃—C₆H₄—CH₃, o-H); Elemental analysis: C: 57.44, H: 8.15, N: 5.43, S: 4.14 (Calculated); C; 57.01, H: 8.28, N: 5.31, S: 4.01 (Found).

### Example 11: Preparation of 2b

Tosylchitin 2 (1 g) with degree of tosylation ˜50% was first dissolved in N,N-dimethylacetamide (DMAc) (35 mL) in sealed screw-top pressure tube. To the solution N,N-dimethyltetradecyamine (7.3 mL) was added and the reaction mixture was heated at 120° C. for 96 h. After the reaction is over, diethyl ether was added in excess (150 mL) to precipitate the product, filtered through a sintered glass funnel and was washed repeatedly with diethyl ether to give white colored compound with 100% degree of quaternization with respect to tosylated group (75-80% yield). FT-IR: υ 3410 cm⁻¹ (OH str.), 2950 cm⁻¹ (—CH₂— assym. str.), 2850 cm⁻¹ (—CH₂— sym. str.), 1675 cm⁻¹ (Amide I, C═O str.), 1630 cm⁻¹ (phenylene), 1560 cm⁻¹ (Amide II, NH ben.), 1470 cm⁻¹ (—CH₂-scissor), 1378 cm⁻¹ (SO₂, asymmetric), 1168 cm⁻¹ (SO₂, symmetric); ¹HNMR: (DMSO-d₆, 400 MHz): δ 0.880 (t, —CH₃(CH₂)₁₁—N⁺(CH₃)₂—, 3H), 1.248 (m, —CH₃(CH₂)₉CH₂CH₂—N⁺(CH₃)₂—, 18H), 1.706 (m, —CH₃(CH₂)₉CH₂CH₂—N⁺(CH₃)₂—, 2H), 1.783 (s, —NHCOCH₃), 2.198 (s, SO₃—C₆—H₄—CH₃), 3.123-4.906 (m, Cell-H and —CH₃(CH₂)₉CH₂CH₂N⁺(CH₃)₂—), 7.104 (d, SO₃—C₆H₄—CH₃, m-H), 7.520 (d, SO₃—C₆H₄—CH₃, o-H); Elemental analysis: C: 58.43, H: 8.36, N: 5.24, S: 3.99 (Calculated); C: 58.20, H: 8.45; N: 5.19, S: 3.85 (Found).

### Example 12: Preparation of 2c

Tosylchitin 2 (1 g) with degree of tosylation ˜50% was first dissolved in N,N-dimethylacetamide (DMAc) (35 mL) in sealed screw-top pressure tube. To the solution N,N-dimethylhexadecyamine (8.1 mL) was added and the reaction mixture was heated at 120° C. for 96 h. After the reaction is over, diethyl ether was added in excess (150 mL) to precipitate the product, filtered through a sintered glass funnel and was washed repeatedly with diethyl ether to give white colored compound with 100% degree of quaternization with respect to tosylated group (75-80% yield). FT-IR: υ 3418 cm⁻¹ (OH str.), 2928 cm⁻¹ (—CH₂— assym. str.), 2858 cm⁻¹ (—CH₂— sym. str.), 1678 cm⁻¹ (Amide I, C═O str.), 1632 cm⁻¹ (phenylene), 1562 cm⁻¹ (Amide II, NH ben.), 1465 cm⁻¹ (—CH₂-scissor), 13.71 cm⁻¹ (SO₂, asymmetric), 1167 cm⁻¹ (SO₂, symmetric); ¹HNMR: (DMSO-d₆, 400 MHz): δ 0.848 (t, —CH(CH₂)₁₁—N⁺(CH₃)₂—, 3H), 1.282 (m, —CH₃(CH₂)₉CH₂CH₂—N⁺(CH₃)₂—, 18H), 1.646 (m, —CH₃(CH₂)₉CH₂CH₂—N⁺(CH₃)₂—, 2H), 1.863 (s, —NHCOCH₃), 2.248 (s, SO₃—C₆H₄—CH₃), 3.068-4.906 (m, Cell-H and —CH₃(CH₂)₉CH₂CH₂—N⁺(CH₃)₂—), 7.028 (d, SO₃—C₆H₄—CH₃, m-H), 7.504 (d, SO₃—C₆H₄—CH₃, o-H); Elemental analysis: C: 59.35, H: 8.56, N: 5.06, S: 3.86 (Calculated); C: 58.95, H: 8.68, N: 4.98, S: 3.79 (Found).

### Example 13: Preparation of 3a

Tosylchitin 3 (1.0 g) with different degree of tosylation ˜60% was first dissolved in N,N-dimethylacetamide (DMAc) (40 mL) in sealed screw-top pressure tube. To the solution N,N-dimethyldodecyamine (7.1 mL) was added and the reaction mixture was heated at 120° C. for 96 h. The product was then precipitated with excess diethyl ether (150 mL), filtered through a sintered glass funnel and washed repeatedly with diethyl ether. Yellowish white colored compounds were obtained with 100% degree of quaternization with respect to tosylated group (75-80% yield). FT-IR: υ 3424 cm⁻¹ (OH str.), 2922 cm⁻¹ (—CH₂— assym. str.), 2852 cm⁻¹ (—CH₂— sym. str.), 1684 cm⁻¹ (Amide I, C═O str.), 1630 cm⁻¹ (phenylene), 1560 cm⁻¹ (Amide II, NH ben.), 1470 cm⁻¹ (—CH₂-scissor), 1382 cm⁻¹ (SO₂, asymmetric), 1170 cm⁻¹ (SO₂, symmetric); ¹HNMR: (DMSO-d₆, 400 MHz): δ 0.878 (t, —CH₃(CH₂)₁₁—N⁺(CH₃)₂—, 3H), 1.282 (m, —CH₃(CH₂)₉CH₂CH₂—N⁺(CH₃)₂—, 18H), 1.686 (m, —CH₃(CH₂)₉CH₂CH₂—N⁺(CH₃)₂—, 2H), 1.874 (s, —NHCOCH₃), 2.232 (s, SO₃—C₆H₄—CH₃), 3.078-4.924 (m, Cell-H and —CH₃(CH₂)₉CH₂CH₂—N⁺(CH₃)₂—), 7.054 (d, SO₃—C₆H₄—CH₃, m-H), 7.484 (d, SO₃—C₆H₄—CH₃, o-H); Elemental analysis: C: 58.41, H: 8.32, N: 5.29, S: 4.54 (Calculated); C: 58.32, H: 8.48, N: 5.18, S: 4.50 (Found).

### Example 14: Preparation of 3b

Tosylchitin 3 (1.0 g) with different degree of tosylation ˜60% was first dissolved in N,N-dimethylacetamide (DMAc) (40 mL) in sealed screw-top pressure tube. To the solution N,N-dimethyltetradecyamine (8.1 mL) was added and the reaction mixture was heated at 120° C. for 96 h. The product was then precipitated with excess diethyl ether (150 mL), filtered through a sintered glass funnel and washed repeatedly with diethyl ether. Yellowish white colored compound were obtained with 100% degree of quaternization with respect to tosylated group (75-80% yield). FT-IR: υ 3419 cm⁻¹ (OH str.), 2925 cm⁻¹ (—CH₂— assym. str.), 2851 cm⁻¹ (—CH₂— sym. str.), 1676 cm⁻¹ (Amide I, C═O str.), 1630 cm⁻¹ (phenylene), 1558 cm⁻¹ (Amide II, NH ben.), 1476 cm⁻¹ (—CH₂— scissor), 1380 cm⁻¹ (SO₂, asymmetric), 1174 cm⁻¹ (SO₂, symmetric); ¹HNMR: (DMSO-d₆, 400 MHz): δ 0.890 (t, —CH₃(CH₂)₁₁—N⁺(CH₃)₂—, 3H), 1.282 (m, —CH₃(CH₂)₉CH₂CH₂—N⁺(CH₃)₂—, 18H), 1.658 (m, —CH₃(CH₂)₉CH₂CH₂—N⁺(CH₃)₂—, 2H), 1.882 (s, —NHCOCH₃), 2.278 (s, SO₃—C₆H₄—CH₃), 3.052-4.956 (m, Cell-H and —CH₃(CH₂)₉CH₂CH₂—N⁺(CH₃)₂—), 7.102 (d, SO₃—C₆H₄—CH₃, m-H), 7.544 (d, SO₃—C₆H₄—CH₃, o-H); Elemental analysis: C: 59.45, H: 8.55, N: 5.09, S: 4.36 (Calculated); C: 59.36, H: 8.60, N: 4.98, S: 4.29 (Found).

### Example 15: Preparation of 3c

Tosylchitin 3 (1.0 g) with different degree of tosylation ˜60% was first dissolved in N,N-dimethylacetamide (DMAc) (20 mL) in sealed screw-top pressure tube. To the solution N,N-dimethylhexadecyamine (8.9 mL) was added and the reaction mixture was heated at 120° C. for 72 h. The product was then precipitated with excess diethyl ether (150 mL), filtered through a sintered glass funnel and washed repeatedly with diethyl ether. Yellowish white colored compound were obtained with 100% degree of quaternization with respect to tosylated group (75-80% yield). FT-IR: υ 3415 cm⁻¹ (OH str.), 2928 cm⁻¹ (—CH₂— assym. str.), 2850 cm⁻¹ (—CH₂— sym. str.), 1676 cm⁻¹ (Amide I, C═O str.), 1635 cm⁻¹ (phenylene), 1562 cm⁻¹ (Amide II, NH ben.), 1465 cm⁻¹ (—CH₂— scissor), 1375 cm⁻¹ (SO₂, asymmetric), 1164 cm⁻¹ (SO₂, symmetric); ¹HNMR: (DMSO-d₆, 400 MHz): δ 0.895 (t, —CH₃(CH₂)₁₁—N⁺(CH₃)₂—, 3H), 1.278 (m, —CH₃(CH₂)₉CH₂CH₂—N⁺(CH₃)₂—, 18H), 1.645 (m, —CH₃(CH₂)₉CH₂CH₂—N⁺(CH₃)₂—, 2H), 1.848 (s, —NHCOCH₃), 2.278 (s, SO₃—C₆H₄—CH₃), 3.046-4.847 (m, Cell-H and —CH₃(CH₂)₉CH₂CH₂—N⁺(CH₃)₂—), 7.012 (d, SO₃—C₆H₄—CH₃, m-H), 7.501 (d, SO₃—C₆H₄—CH₃, o-H); Elemental analysis: C: 60.42, H: 8.75, N: 4.90, S: 4.20 (Calculated); C: 60.38, H: 8.80, N: 4.82, S: 4.15 (Found).

Various chitin derivatives were prepared: 1a-1c, 2a-2c and 3a-3c by reacting each of Tsch 1 (DS ˜40%), Tsch 2 (DS ˜50%) and Tsch 3 (DS ˜60%) with N,N-dimethyldodecylamine, N,N-dimethyltetradecylamine and N,N-dimethylhexadecylamine respectively at 120° C. for 96 h. These derivatives were characterized by ¹H-NMR, FT-IR, and elemental analysis. All the tosyl groups in the tosylchitins were completely quaternized (Table 3). The complete quaternization was confirmed from ¹H-NMR as the NMR spectra clearly revealed the presence of only two doublet aromatic peaks at 7.093 and 7.514 ppm corresponding to protons of benzene ring in tosylate anion of quaternized chitin derivatives (The signals at δ 7.112 and 7.452 representing the aromatic protons of the covalently attached tosyl groups disappeared completely after quaternization).

TABLE 3
Table 3 describes the properties of 1a-1c, 2a-2c and 3a-3c

| | Tosyl chitin | | N,N-dimethyl | Degree of |
|---|---|---|---|---|
| | used for | | amine ratio/ | quaternization |
| Sample | quaternization | R′ | Tosylated sugar | (DQ) |
| 1a | tosylchitin 1 | —C₁₂H₂₅ | 10 | ~40 |
| 1b | tosylchitin 1 | —C₁₄H₂₉ | 10 | ~40 |
| 1c | tosylchitin 1 | —C₁₆H₃₃ | 10 | ~40 |
| 2a | tosylchitin 2 | —C₁₂H₂₅ | 10 | ~50 |
| 2b | tosylchitin 2 | —C₁₄H₂₉ | 10 | ~50 |
| 2c | tosylchitin 2 | —C₁₆H₃₃ | 10 | ~50 |
| 3a | tosylchitin 3 | —C₁₂H₂₅ | 10 | ~60 |
| 3b | tosylchitin 3 | —C₁₄H₂₉ | 10 | ~60 |
| 3c | tosylchitin 3 | —C₁₆H₃₃ | 10 | ~60 |

### Example 16: Solubility of the Chitin Derivatives

A small portion (10 mg) of all the chitin derivatives were added in 1 mL of various organic solvents (chloroform, dichloromethane, methanol, ethanol, butanol, dimthylformamide, dimethyl sulfoxide, tetrahydrofuran) and vortexed for about 10 min and observed visually to check the solubility. The solubility limit of the derivatives was also determined visually after vortexing for 10-15 min of different amounts (10, 20, 50, and 100 mg) in 1 ml of solvent. However, to test water solubility, 10 mg of the chitin derivatives in 1 ml of water was vortexed for 5 min and kept for 24 h. The aqueous part was filtered and subjected to freeze drying. ¹HNMR spectra were recorded with the freeze dried sample in deurerioted methanol (CD₃OD) to check the solubility of the derivatives in aqueous media. It was found that 1a is partially soluble in water while 1b-1c, 2a-2b, and 3a-3c are completely insoluble in water (Table 4). The insolubity in aqueous media and solubility in organic solvents such as methanol indicate that these polymers can simply be coated onto the surface from their organic solutions to prepare antibacterial coatings.

TABLE 4
Solubility of the chitin derivatives

| Chitin | | | | | | | | | |
|---|---|---|---|---|---|---|---|---|---|
| derivatives | H₂O | CHCl₃ | CH₂Cl₂ | MeOH | EtOH | n-BuOH | DMF | DMSO | THF |
| 1a | ± | − | − | + | + | − | + | + | − |
| 1b | − | − | − | + | + | − | + | + | − |
| 1c | − | − | − | + | ± | − | + | + | − |
| 2a | − | − | − | + | ± | − | + | + | − |
| 2b | − | − | − | + | ± | − | + | + | − |
| 2c | − | − | − | + | ± | − | + | + | − |
| 3a | − | − | − | + | ± | − | + | + | − |
| 3b | − | − | − | + | ± | − | + | + | − |
| 3c | − | − | − | + | ± | − | + | + | − |

+ = Soluble;
− = Insoluble;
± = Partially soluble;

### Example 17; Preparation of the Polymer Film/Coating

Chitin derivatives were first dissolved in methanol (50 mg/mL) and the solutions were serially diluted. 20 μL of the solutions of different concentrations were added into wells of a 96-well plate. The solution was first air-dried and finally dried in vacuum oven to make a film onto the flat-bottom surface of wells of 96-well plate. Each concentration was applied in triplicate for all the samples to study the antibacterial activity. In order to make films on surfaces such as microscopic glass slide, methanolic solutions of polymer (350 μL) were spin coated onto the glass surface by using a spin coater. In order to show the potential of the newly synthesized chitin derivatives to be used in medical devices and implants as antibacterial coating, thin films of these derivatives were prepared along with the medically relevant polymers such PLA, PMMA, or PLGA. To prepare thin films, mixed solutions (350 μL) obtained from the solutions of these medically relevant polymers in chloroform and methanolic solution of the chitin derivatives (CHCl₃: MeOH=9:1) were spin coated onto the glass surface and dried in air. For the biodegradation study, thin films of the chitin derivatives were prepared on microscopic cover glass. To prepare the film, chitin derivative (2c) was dissolved in DMSO (50 mg/mL) and 50 μL of the solution was drop casted on cover glass and dried in oven at 70° C. overnight.

### Example 18: In-Vitro Antibacterial Activity of the Chitin Derivatives

To determine the antibacterial activity of the synthesized polymers, 200 μL of 10⁵ CFU/mL of bacteria were added to the wells of 96-well plate coated with the chitin derivatives with different amounts. Two controls were made: in one control no solvent was added to the wells (blank wells) and the other one is solvent-dried well. The plates were then placed in an incubator and were incubated at 37° C. for 24 h. After incubation, the optical density (O.D.) value was recorded using TECAN (Infinite series, M200 pro) Plate Reader. Each concentration had triplicate values and the whole experiment was done at least twice and the minimum inhibitory amount was determined by taking the average of triplicate O.D. values for each concentration and plotting it against concentration. The data was then subjected to sigmoidal fitting. From the curve the minimum inhibitory amount was determined, as the point in the curve where the O.D. was similar to that of control having no bacteria. The minimum inhibitory amount (μg/well as obtained after drying the solvent) was converted into the minimum inhibitory concentration (MIC) (μg/mL) by considering the fact that the coated amount in a well is present in 200 μL of the bacterial media. Subsequently, the amount present in a well was multiplied by a factor of 5 to get MIC as μg/mL. In order to determine the minimum bactericidal concentration (MBC), aliquots from wells (20 μL) that appeared to have little or no cell growth were plated on agar plates to distinguish between bacteriostatic or bactericidal effects. These were incubated at 37° C. for 24 h, and then colonies were observed.

These derivatives, when coated into the wells of 96-well plate, were found to be highly antibacterial against S. aureus, a Gram-positive bacterium as well as E. coli, a Gram negative bacterium (FIG. 1). 1c and 2c were found to be most active against both types of bacteria (each 10 μg/mL for S. aureus and 312 and 156 μg/mL for E. coli respectively). However, these derivatives were found to be more active against S. aureus compared to E. coli. Interestingly, 3a-3c were less active or inactive upto the tested concentration (5000 μg/mL) against E. coli (FIG. 1B). This might be due to the highly hydrophobic nature and hence improper balance of hydrophobicity/hydrophilicity which is necessary for having the activity. When tested against drug-resistant bacteria, these polymers showed high activity against methicillin-resistant S. aureus (MRSA), vancomycin-resistant enterococci (VRE), and β-lactam-resistant K. pneumoniae (Table 5). Like sensitive bacteria, these derivatives were found to be more active against drug resistant Gram-positive bacteria (MRSA and VRE) compared to drug resistant Gram-negative bacteria (K. pneumoniae).

The present disclosure provides a hydrophobically modified cationic chitin derivatives using facile synthetic methodology. These derivatives showed strong, broad-spectrum antibacterial activity. These derivatives being insoluble in water and soluble in organic solvents can easily be coated onto any surfaces by non-covalent modification of the surface. Thus, this strategy can be a promising approach to develop highly effective antimicrobial coatings.

TABLE 5
Minimum inhibitory concentrations of the chitin
derivatives against drug resistant bacteria

| | MIC (μg/mL) | | |
|---|---|---|---|
| Chitin derivatives | MRSA | VRE | K. pneumoniae |
| 1b | 78 | 78 | >5000 |
| 1c | 39 | 39 | 2500 |
| 2a | 78 | 156 | >5000 |
| 2b | 39 | 78 | >2500 |
| 2c | 20 | 20 | 1250 |
| 3a | 1250 | 1250 | >5000 |
| 3b | 312 | 312 | >5000 |
| 3c | 312 | 156 | >5000 |

MBC values were determined by plating about 20 μL of the solution containing bacteria after 24 h of treatment and later counting the colonies after their development on suitable agar plate. MBC values show that these derivatives act not only as bacteriostatic but also bactericidal as well (Table 6).

TABLE 6
Minimum bactericidal concentrations of the chitin derivatives
against drug-sensitive and drug-resistant bacteria

| Chitin | MBC (μg/mL) | | | | |
|---|---|---|---|---|---|
| derivatives | S. aureus | E. coli | MRSA | VRE | K. pneumoniae |
| 1b | 40 | >5000 | 78 | 78 | >5000 |
| 1c | 10 | 5000 | 39 | 39 | >2500 |
| 2a | 156 | 5000 | 78 | 156 | >5000 |
| 2b | 78 | 2500 | 39 | 78 | >2500 |
| 2c | 10 | 156 | 20 | 20 | >1250 |
| 3a | 312 | >5000 | 1250 | 1250 | >5000 |
| 3b | 312 | >5000 | 312 | 312 | >5000 |
| 3c | 156 | >5000 | 312 | 156 | >5000 |

### Example 19: Antibacterial Kinetics

Well plate was coated with the polymers 1c and 2c following the same coating procedure at two different concentrations: MIC and 6×MIC. A quantity of 200 μL of a solution containing approximately 4.9×10⁵ CFU/mL of S. aureus in nutrient broth and 5.1×10⁵ CFU/mL in Luria-Bertani (LB) broth were added, and the plates were kept in an incubated shaker at 37° C. The initial time of addition of the bacteria to the wells was taken as zero, and 10 μL aliquots were withdrawn from each of the wells at set time intervals. These aliquots were added immediately to 90 μL of 0.9% saline. These solutions were further diluted by a factor of 10, and 20 μL of all the dilutions was plated on nutrient or LB agar plates immediately. The plates were incubated at 37° C. for 24 h; and bacterial colonies were counted. A plot of CFU (Log₁₀ CFU/mL) versus time was then plotted.

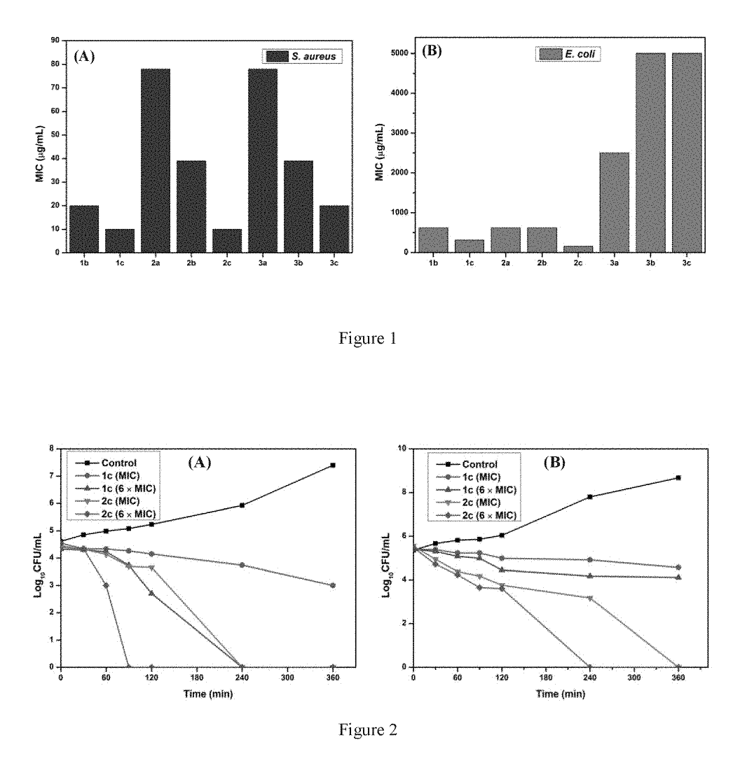

The kinetics of antibacterial activity of the chitin derivatives (1c and 2c) towards both S. aureus and E. coli were investigated in order to establish the rate of antibacterial action of these polymers (FIG. 2). It was found that the polymer 2c at 6×MIC killed S. aureus at 90 min whereas 1c at 6×MIC killed at 240 min. However, 2c at MIC (which is MBC as well) killed S. aureus at 240 min whereas 1c at MIC (as well as MBC) was found to be bacteriostatic upto 360 min (6 h) (FIG. 2A). On the other hand, 2c at 6×MIC killed E. coli at 240 min whereas 1c at 6×MIC was found to be bacteriostatic even upto 360 min (6 h). 2c at MIC (as well as MBC) killed E. coli at 360 min whereas 1c at MIC (MBC=5000 ug/mL) was found to be bacteriostatic upto 360 min (6 h) (FIG. 2B). These results suggested that 2c having higher degree of quaternization than 1c (although the MIC values are same for both the compounds against S. aureus) killed Gram-positive bacteria more rapidly. However, in case of Gram-negative bacteria, 2c being more active (MIC values for 2c and 1c are 156 and 312 μg/mL) showed faster killing rate. These results further indicated that an optimum hydrophobic/hydrophilic balance is required to have better activity and faster killing rate.

### Example 20: Antibacterial Activity by Spray Method

In order to evaluate the ability of these polymers to serve as antibacterial paint and to simulate the natural deposition of airborne bacteria as well as contact deposition of bacteria onto surface, antibacterial activity of the polymers were determined by coating the polymers onto surface and then spraying the bacteria onto coated surface. Antibacterial activity of the chitin derivatives was also tested similarly by coating the derivatives along with polylactic acid (PLA) in order to show the utility of these derivatives to be used as antibacterial agents in the biomedical field. Bacteria were grown for 6 h in the suitable nutrient media at 37° C. under constant shaking. The 1 mL of the 6 h grown bacteria was centrifuged down at a speed of 12000 rpm for 1 min. The bacterial pellet was then washed twice with 1×PBS (pH−7.4). Final concentration of the bacterial solution was then adjusted to 10⁷ cfu/mL for S. aureus and 10⁶ cfu/mL for E. coli and the volume was made to 10 mL. The bacterial solution was then sprayed onto the non-coated, PLA coated (as controls) and coated glass slides (2.5 cm×5.5 cm) at a spray rate of approximately 10 mL/min. The sprayed slides were carefully transferred into a petridish and were allowed to get dried. A slab of nutrient agar was placed onto the glass slide and the pertidish was sealed and kept at 37° C. till visible colonies developed. The coated and non-coated slides were imaged using a Cell Biosciences Gel Documentation instrument. Images were captured under white light and processed using Alpha-imager software.

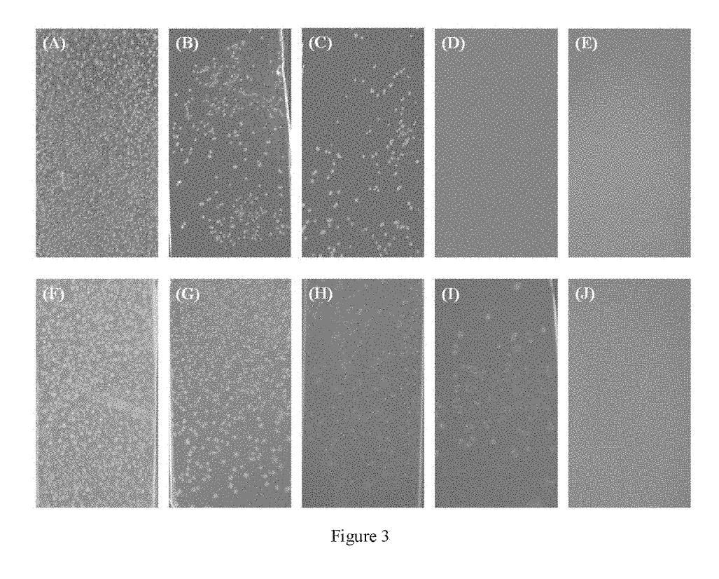

Bacterial growth was seen on non-coated glass surfaces as indicated by the presence of colonies whereas no/lesser colony was observed on chitin derivative (2c) coated surfaces (FIG. 3). Polymer 2c killed completely (at least 5-log reduction with respect to control) Gram-positive bacterium S. aureus at 16 μg/cm² (FIG. 3D) and at higher amount 32 μg/cm² (FIG. 3E) whereas at 4 μg/cm² (FIG. 3B) and at 8 μg/cm² (FIG. 3C), bacterial colonies were observed though the number of colonies is much lower as compared to control (FIG. 3A). Polymer 2c, on the other hand, killed completely (at least 4-log reduction with respect to control) Gram-negative bacterium E. coli at 32 μg/cm² (FIG. 3J) whereas at 4 μg/cm² (FIG. 3G), at 8 μg/cm² (FIG. 3H) and at 16 μg/cm² (FIG. 3I), bacterial colonies were observed though the number of colonies is lower as compared to control (FIG. 3F). These results are therefore indicating that the compounds disclosed in the present disclosure could be used as antibacterial paint in various biomedical and house-hold applications.

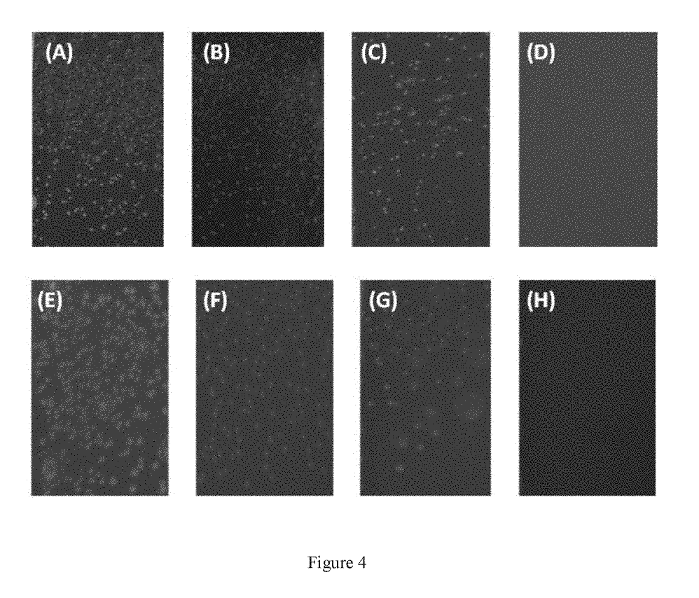

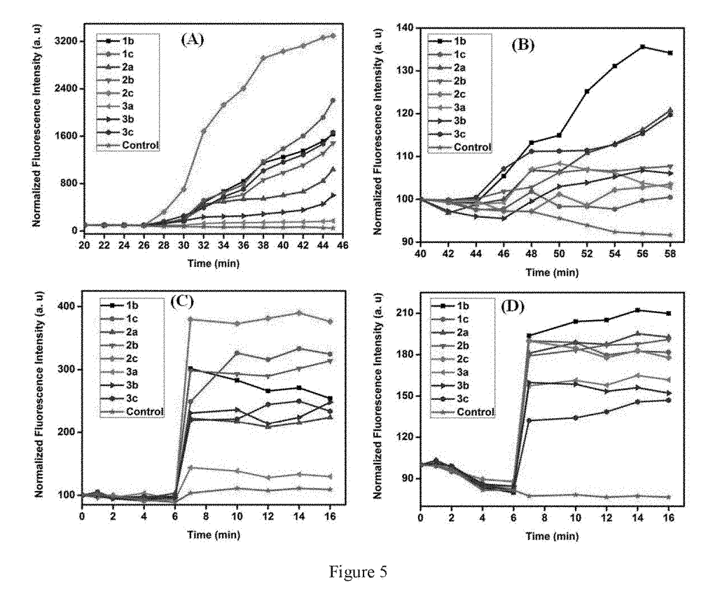

The compound when coated onto the glass surface along with medically relevant polymer and bacteria were sprayed also showed antibacterial activity against both S. aureus and E. coli (FIG. 4). Complete activity (at least 5-log reduction with respect to control) was observed against S. aureus when the amount of (PLA+2c) coated onto glass surface was (255+16) μg/cm² (FIG. 4D) whereas (255+4) μg/cm² (FIG. 4B), (255+8) μg/cm² (FIG. 4C) coated surfaces showed bacterial growth though the number of colonies is much lower as compared to only PLA coated surface (FIG. 4A). On the other hand, complete activity (at least 4-log reduction with respect to control) was observed against E. coli when the amount of (PLA+2c) coated onto glass surface was (255+32) μg/cm² (FIG. 4H) whereas (255+8) μg/cm² (FIG. 4F), (255+16) μg/cm² (FIG. 4G) coated surfaces showed bacterial growth though the number of colonies is lower as compared to only PLA coated surface (FIG. 4E). Thus at 6.25 and 12.5 wt % loading of 2c with respect to PLA showed complete activity. These results are indicating that these polymers could be used as potential antibacterial coatings in various biotechnological/biomedical applications and also be used to develop self-defensive biomaterials.

### Example 21: Antibacterial Activity in Mammalian System

Blood (sodium heparin as anticoagulant) was donated by healthy human donors. Plasma was isolated by centrifugation of the blood at 3500 rpm for 5 min. Serum was obtained by using serum tube containing human blood and then centrifuging the blood at 3500 rpm for 5 min. Methicillin-resistant S. aureus (MRSA) was grown at nutrient media for 6 h (˜10⁹ CFU/mL). Then the bacteria were diluted in minimum essential medium (MEM) to obtain 2×10⁷ or 10⁶ CFU/mL. Finally, MRSA was diluted in all three mammalian systems to obtain 10⁵ CFU/mL in 50% serum, 50% plasma, and 90% blood (required volume of MEM containing MRSA and various mammalian systems were mixed to obtain 50% serum, 50% plasma, and 90% blood having 10⁵ CFU/mL of MRSA). To determine the antibacterial activity of the synthesized polymers 1c, and 2c in mammalian system, 200 μL of 50% serum, 50% plasma, and 90% blood containing 10⁵ CFU/mL of MRSA were added to the wells of 96-well plate coated with the chitin derivatives with different amounts. Likewise the MIC experiment, two controls were made: in one control no solvent was added to the wells (blank wells) and the other one is solvent-dried well. The plates were then placed in an incubator at 37° C. for 18 h or 24 h. After incubation, visual turbidity of the coated well plate containing mammalian systems with MRSA was noted and the optical density (OD) value was recorded using TECAN (Infinite series, M200 pro) Plate Reader for serum and plasma. Likewise the MIC experiment, the minimum inhibitory amount (μg/well as obtained after drying the solvent) was converted into the minimum inhibitory concentration (MIC) (μg/mL) by considering the fact that the coated amount in a well is present in 200 μL of the mammalian systems. As for the blood, inhibitory concentration cannot be obtained, so minimum bactericidal concentration (MBC) was determined. In order to determine MBC, aliquots from wells (20 μL) of the 96-well plate containing blood as the experimental media that appeared to have little or no cell growth were plated on agar plates. These were incubated at 37° C. for 24 h, and then colonies were observed.

Both the polymers retained their antibacterial activity even after exposure to complex mammalian fluids (Table 7). 1c and 2c were found to have same or 2-fold MIC values in 50% human serum or plasma as obtained in growth medium: 10 μg/mL and 20 μg/mL. This further proved that the polymers did not lose their antibacterial activity in the presence of growth medium containing 50% human serum or plasma. Interestingly, it was found that these polymers retained their antibacterial activity even in 90% blood. The minimum bactericidal concentrations (MBC) of 1c and 2c were found to be 2- or 4-fold higher than that of the growth media. As the blood is very complex media containing negatively charged proteins, macromolecule, etc. which presumably might interact with the positively charged chitin derivatives thereby deactivating the chitin polymers to some extent toward membrane disruption of bacteria? The above results suggest that the newly prepared chitin derivatives can be used in-vivo as antimicrobial coatings.

TABLE 7
Minimum inhibitory concentrations of the chitin
derivatives against MRSA in mammalian systems

| | MIC/MBC in | MIC in 50% | | MBC in 90% |
|---|---|---|---|---|
| | TSB Broth | Serum | MIC in 50% | Blood |
| Sample | (μg/mL) | (μg/mL) | Plasma (μg/mL) | (μg/mL) |
| 1c | 10/10 | 20 | 20 | 39 |
| 2c | 10/10 | 10 | 10 | 39 |

### Example 22: Mechanism of Action (Cytoplasmic Membrane Depolarization Assay)

Midlog phase bacterial cells (S. aureus and E. coli) were harvested, washed with 5 mM HEPES and 5 mM glucose and resuspended in 5 mM glucose, 5 mM HEPES buffer and 100 mM KCl solution in 1:1:1 ratio (10⁸ CFU/mL). For the cytoplasmic membrane depolarization assay, 150 μL of the bacterial suspension and 50 μL of 8 μM DiSC₃(5) were added in black 96-well plate. The fluorescence of the dye was allowed to quench for 20 min for S. aureus and 40 min for E. coli respectively. Additionally, 0.2 mM EDTA was used in case of E. coli to allow the dye uptake through the outer membrane. Then, the dye containing bacterial suspension was added in another 96-well plate (black plate, clear bottom with lid) coated with the chitin derivatives and fluorescence intensity was measured at every 2 minutes interval for next 25 min using TECAN plate reader with the following excitation and emission wavelength: excitation wavelength; excitation wavelength: 622 nm (slit width: 10 nm) and emission wavelength: 670 nm (slit width: 20 nm). The 96-well plates were coated following the similar coating procedure as mentioned previously to give polymer concentrations of 200 μg/mL for S. aureus and 2000 μg/mL for E. coli.

### Example 23: Mechanism of Action (Intracellular K⁺ Ion Leakage Assay)

Midlog phase bacterial cells (S. aureus and E. coli) were harvested, washed twice with 10 mM HEPES (pH 7.2) and 0.5% wt/vol glucose and were resuspended in the same amount of 10 mM HEPES (pH 7.2) and 0.5% wt/vol glucose. The bacterial suspension (150 μL, 10⁸ CFU/mL) was placed in black 96-well plate. The fluorescence of the bacterial suspension was measured and allowed to stabilize for 2 minutes at room temperature before the addition of PBFI-AM dye (50 μL, 4 μM). Fluorescence was recorded for an additional 2 min to establish a baseline signal before the addition of dye containing bacterial suspension to polymer coated wells of a 96-well plate (black plate, clear bottom with lid). Then, the fluorescence intensity was monitored at every 2 minutes interval for next 10 minutes using TECAN plate reader with the following excitation and emission wavelength; excitation wavelength: 346 nm (slit width: 10 nm) and emission wavelength: 505 nm (slit width: 20 nm). The 96-well plates were coated following the similar coating procedure as mentioned previously to give polymer concentrations of 200 μg/mL for S. aureus and 2000 μg/mL for E. coli.

In order to evaluate the mechanism of antibacterial action of the cationic polymers (1b-1c, 2a-2c and 3a-3c), both membrane depolarization and intracellular potassium ion leakage were performed. All the polymers were found to depolarize the membrane potential (FIGS. 5A and 5B against S. aureus and E. coli respectively) and were able to cause the leakage of intracellular potassium ions (FIGS. 5C and 5D against S. aureus and E. coli respectively). Comparing the membrane depolarization ability of polymers containing the same alkyl chain length (—C₁₆H₃₃) but varying DQ (1c with 40% DQ, 2c with 50% DQ and 3c with 60% DQ), it was observed that the polymer 2 (with 50% DQ) was the most efficient. Interestingly, in case of polymers with same DQ but varying alkyl chain length (2a, 2b and 2c all with 50% DQ), polymer 2c (with —C₆H₃₃ alkyl chain) was the most effective in depolarizing the S. aureus cell membrane (FIG. 5A). On performing a similar study with E. coli it was observed that in case of polymers with constant chain length but variable DQ (1b, 2b and 3b), 1b (with DQ=40%) was the most efficient; whereas on keeping the same DQ and varying chain length (2a, 2b and 2c), 2a (with —C₂H₂₅ alkyl chain) was the most effective (FIG. 5B). Polymers with the higher DQ (3a-3c) were found to be less effective in dissipating membrane potential of both the bacteria. Similar results were obtained for the intracellular potassium ion leakage assays against both S. aureus (FIG. 5C) and E. coli (FIG. 5D).

### Example 24: Mechanism of Action (Fluorescence Microscopy)

Bacteria were grown in suitable media for 6 h. Bacterial suspension (200 μL, 10⁹ and 10⁸ CFU/mL for S. aureus and E. coli respectively) were added to the well of 96 well plate coated by chitin derivative (2c, 6×MIC). A control was made similarly by adding bacteria in non-coated wells. The 96-well plate was placed in incubator at 37° C. under constant shaking for 4 h. After the incubation, the bacterial suspension was harvested and washed with PBS twice and finally resuspended in 100 μL PBS. Then, 10 μL of the bacterial suspension was combined with 20 μL of a fluorescent probe mixture containing 3.0 μM green fluorescent nucleic acid stain SYTO 9 (Invitrogen, USA) and 15.0 μM red fluorescent nucleic acid stain propidium iodide (PI) (Sigma Aldrich, USA) (1:1 v/v). The mixture was incubated in dark for 15 min and a 5 μL aliquot was placed on a glass slide, which was then covered by a cover slip, sealed and examined under fluorescence microscope. Excitation was done for SYTO 9 at 488 nm and at 543 nm for PI respectively. Emission was collected using a band pass filter for SYTO 9 at 500-550 nm and a long pass filter for PI at 590-800 nm. In all cases, a ×100 objective was used with immersion oil, giving a total magnification of ×1000. Images were captured with a Leica DM 2500 fluorescence microscope.

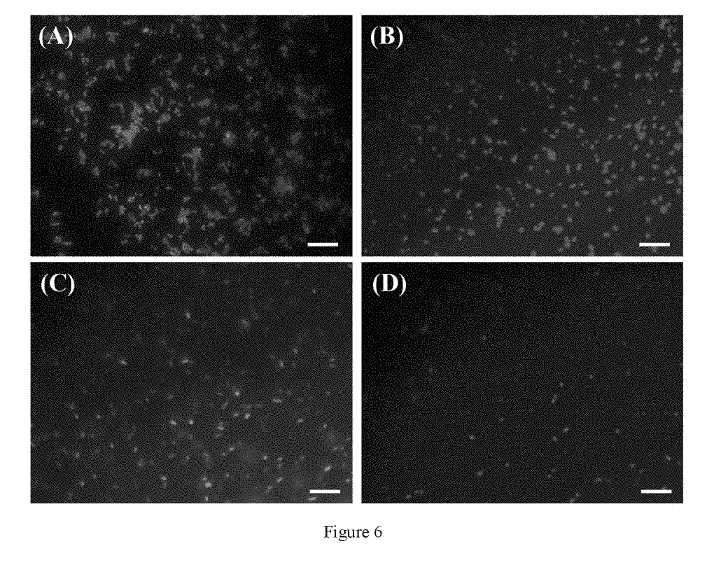

The fluorescence microscopy images showed the cell viability in case of the control samples (untreated) by green fluorescence (FIG. 6A for S. aureus and FIG. 6C for E. coli). On the other hand, images of the cells treated with the polymer 2c coated surface showed complete membrane permeabilization indicated by red fluorescence (FIG. 6C for S. aureus and FIG. 6D for E. coli).

### Example 25: Mechanism of Action (Field Emission Scanning Electron Microscopy)

Bacteria (S. aureus and E. coli) were grown overnight at 37° C. in suitable nutrient media, washed, and prepared as previously described. 200 μL of the bacterial suspension (10⁸ CFU/mL for both S. aureus and E. coli respectively) in media were added to the wells of 96-well plate coated by 2c (6×MIC). Bacteria were incubated at 37° C. for 2 h. After incubation, the bacterial suspension from the wells was transferred to 1 mL eppendrof tube and centrifuged. The bacterial pellet was then resuspended in 30% ethanol and subsequently dehydrated with 50%, 70%, 90%, and 100% ethanol. Finally, the bacteria were resuspended in 90% ethanol and 5 μL of the bacterial suspension in ethanol was drop casted onto silicon wafer and dried at room temperature. The samples were sputter coated with gold prior to imaging using Quanta 3D FEG, FEI field emission scanning electron microscopy.

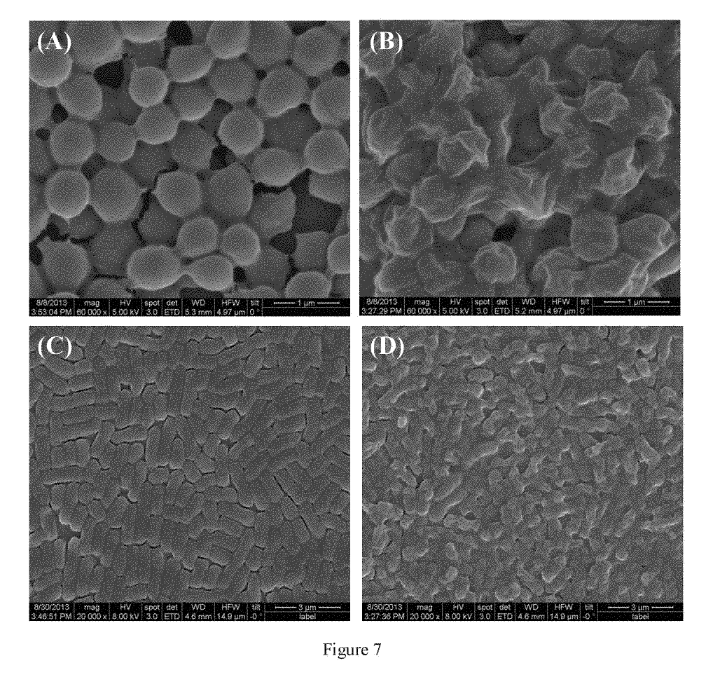

The micrographs for the untreated bacteria showed well-defined morphology and smooth surface characteristic of unperturbed bacteria (FIG. 7A for S. aureus and FIG. 7C for E. coli). In contrast, treated bacteria exhibited profound morphological deformations (FIG. 7B for S. aureus and FIG. 7D for E. coli) which indicated that the hydrophobic cationic polymer interacted with the lipid membrane of bacteria and consequently disrupted the membrane integrity. The membrane-active nature of the compound of Formula I would reduce the propensity of development of bacterial resistance.

### Example 26: Hemolytic Activity

Erythrocytes were isolated from freshly drawn, heparanized human blood and resuspended to 5 vol % in PBS (pH 7.4). In a polymer-coated 96-well plate, 200 μL of erythrocyte suspension (5 vol % in PBS) was added. Two controls were made, one without polymer-coated well and other containing with 1 vol % solution of Triton X-100. The plate was incubated for 1 h at 37° C. The plate was then centrifuged at 3,500 rpm for 5 min, 100 μL of the supernatant from each well was transferred to a fresh microtiter plate, and absorbance at 414 nm was measured. Percentage of hemolysis was determined as (A−A₀) (Aₜₒₜₐₗ−A₀)×100, where A is the absorbance of the test well, A₀ the absorbance of the negative controls, and Aₜₒₜₐₗ the absorbance of 100% hemolysis wells, all at 414 nm.

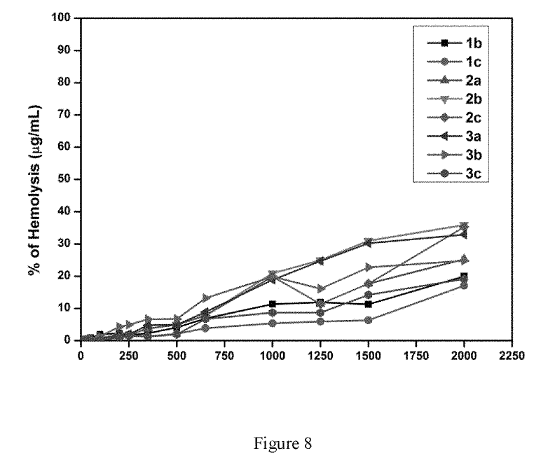

Hemolytic assay showed that these derivatives are non-hemolytic even upto 750 μg/mL. Only 20-30% hemolysis was observed at 2000 μg/mL (FIG. 8). The most active derivatives 1c and 2c showed negligible hemolysis even upto 1000 μg/mL and 10-15% hemolysis at 2000 μg/mL which is 2-100 times more than their MIC values. These results thus indicating that these derivatives are non-hemolytic and therefore can be used as antimicrobial coatings in various bio-medical applications.

### Example 27: Cyotoxicity with Mammalian Cells

The cells (HEK 293) were cultured in Dulbecco's Modified Eagle Medium (DMEM) supplemented with 10% heat-inactivated FBS, 1% penicillin-streptomycin solution and incubated at 37° C. in 5% CO₂. 96 well plates were first coated with the chitin derivative 2c at two different amounts (at low and high MIC respectively). Blank wells and wells in which equal amount of solvent was added and dried, were taken as negative controls. The coated 96-well plate was sterilized by exposing the plate to UV radiation for 10 minutes. After sterilization, 200 μL of growth media containing 10⁴ HEK 293 cells were then seeded onto the coated and uncoated wells. The plate was incubated at 37° C. under a 5% CO₂-95% air atmosphere for 24 h. At the end of the incubation period, bright-field images of the wells containing cells were taken through a 20×objective of Leica DM IL LED microscope.

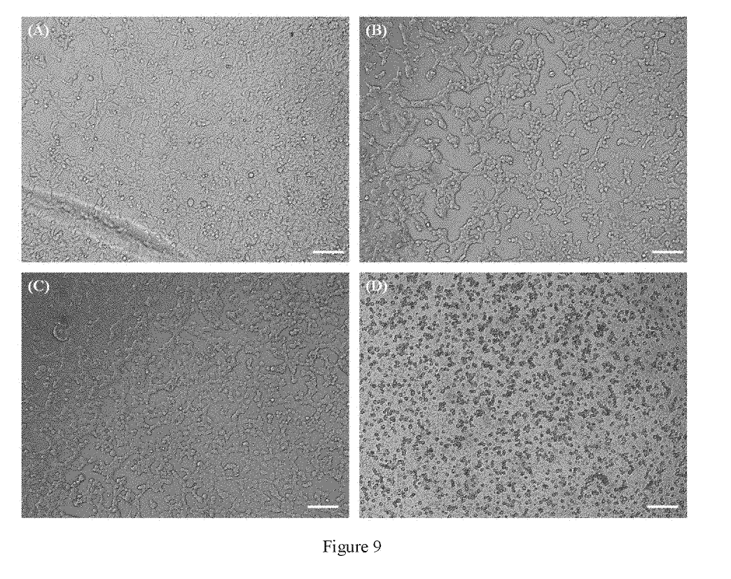

At both low and high concentrations, cells were found to retain their morphology (spindle shape) (FIGS. 9B and 9C) and were almost identical with the untreated cells (FIG. 9A) whereas triton-x treated cells were of completely spherical shape (FIG. 9D). These results however emphasized that the chitin derivatives are non-toxic at their MIC values.

### Example 28: Biodegradation Study by Scanning Electron Microscopy

Microscopic cover glass (13 mm) were coated with the chitin derivative 2c following the coating procedure as described previously. The glasses were placed in a 6-well plate. The media for the biodegradation testing was a sodium acetate buffer solution (50 mmol, pH=5.5). Lysozyme was dissolved in the buffer solution to give an enzyme-solution with an enzyme activity of 29000 units/mL. Then, the coated cover glass was placed in the lysozyme containing buffer solution. For comparison, another reference sample was placed in an enzyme-free buffer-solution. The samples were incubated at 37° C. for 20 days under agitation. The cover glasses were removed from the well plates, washed with buffer and immersed into liquid nitrogen followed by freeze drying in vacuum oven. The films of the chitin derivatives before and after treatment were finally imaged with field emission scanning electron microscopy (FESEM) at 5 kV operating voltage.

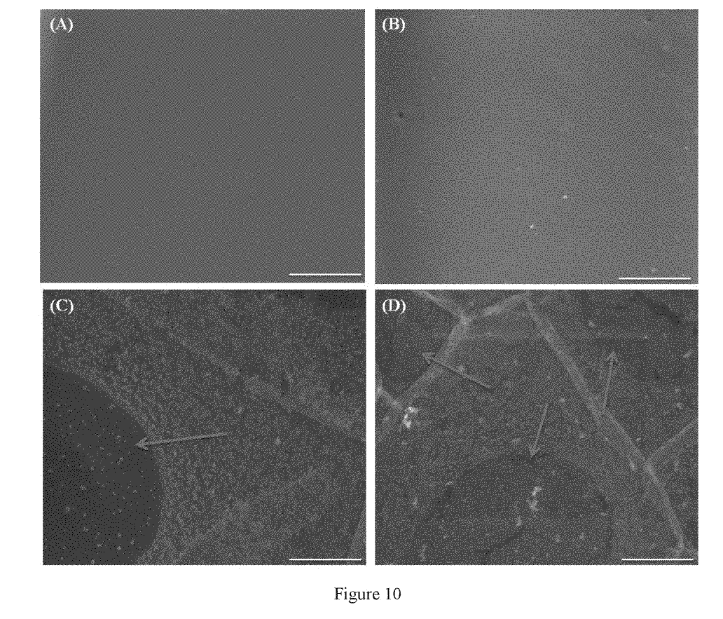

After treating with lysozyme, film of polymer 2c showed porous structures with holes (FIGS. 10C and 10D at day 15 and 20 respectively) whereas treating the polymer with buffer alone left the coating unchanged (FIG. 10B), almost identical to the film without any treatment with buffer (FIG. 10A). These results thus showed that the chitin backbone of these derivatives is susceptible towards lysozyme making them biodegradable and hence suitable for various biomedical applications.

### Example 29: Synthesis of Polymeric Nanocomposites

The polymeric nanocomposites were prepared in-situ from a mixture of solution of chitin derivative (1c) and solution of silver para-toluene sulfonate (AgPTS). The nanocomposites were prepared by adding solution of silver para-toluene sulfonate (AgPTS) in DMSO to a solution of chitin derivative (1c) in methanol at a ratio 1:1 and 1:0.5 (wt/wt of 1c/AgPTS) and the mixture was kept at room temperature for about 48 h. In-situ formation of silver nanoparticles (Ag NPs) was observed within 6 h where the chitin derivatives and/or DMSO act as reducing agents and chitin derivatives as stabilizing agent. As the method of formation is independent of the degree of quaternization and type of quaternary ammonium groups, any polymer from the Formula I of the current patent could be used to synthesize silver nanoparticle in-situ.

The formation of silver nanoparticle was confirmed by UV-visible absorption spectroscopy and transmission electron microscopy (TEM). UV-visible absorption spectroscopy showed the appearance of surface plasmon band for silver nanoparticle at 410 nm (FIG. 11A). TEM images showed that size of these nanoparticles ranges from 20-200 nm (FIG. 11B).

### Example 30: Antibacterial Activity of the Nanocomposites (Spray Method)

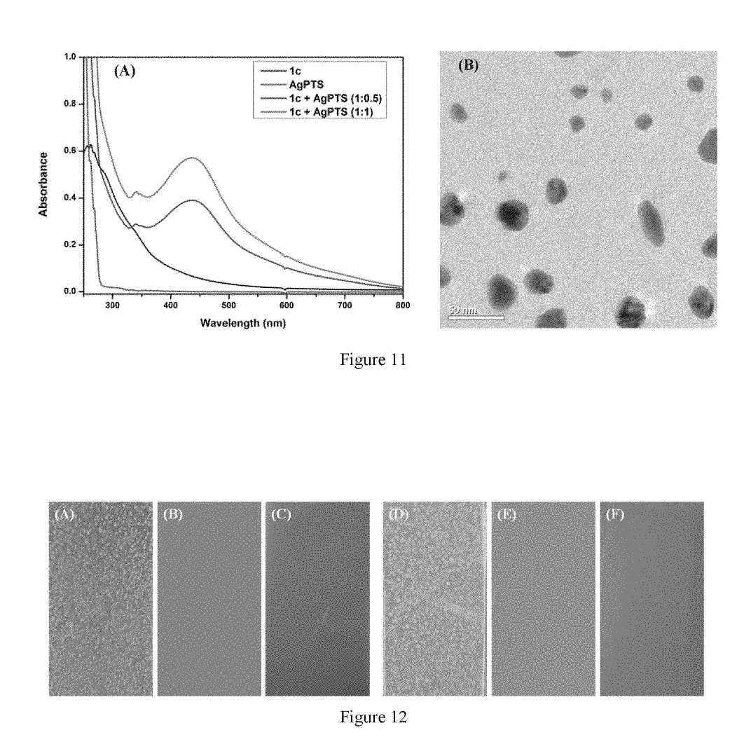

In order to evaluate the ability of these composites to serve as antibacterial paint, glass slides were coated with different amounts of the composites and assayed for antibacterial activity as mentioned previously. Bacterial growth was seen on non-coated glass surface as indicated by the presence of colonies whereas no colony (100% activity, 5-log reduction with respect to control) was observed on composite-coated surfaces (FIG. 12). Notably, the composite coated surfaces showed much higher activity compared to only polymer coated surface against both Gram-positive and Gram-negative bacteria. Polymer 1c coated surfaces showed 100% activity against S. aureus at 30 μg/cm² and against E. coli at 60 μg/cm² respectively. Whereas, the polymeric composite (1c: AgPTS=1:0.5) coated surfaces exhibited 100% activity against S. aureus at (10+5) μg/cm² and against E. coli at (20+10) μg/cm² respectively. These results clearly showed that these polymeric nanocomposites probably act synergistically and are therefore promising candidate as coating materials.

### Example 31: Antibacterial Activity Against Water-Borne Bacteria

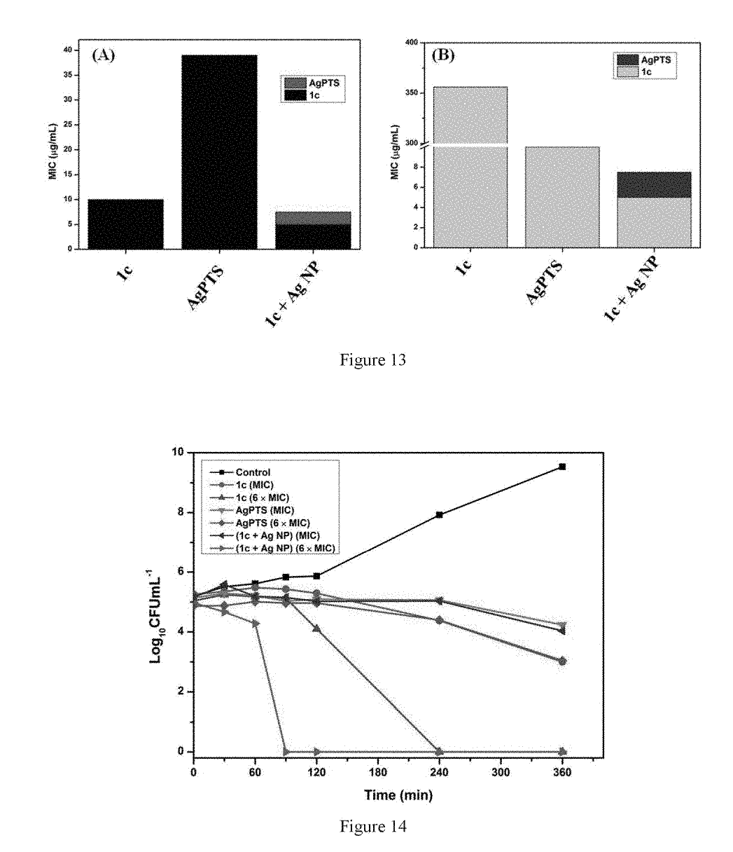

68 Antibacterial activity of the composites was determined against water-borne bacteria by coating the wells of a 96-well plate and adding bacterial suspension (200 μL, 10⁵ CFU/mL), incubating for 24 h at 37° C. Though both 1c and AgPTS are active against both Gram-positive and Gram-negative bacteria, the polymeric nanocomposites showed better activity as compared to the individual components (FIG. 13). For example, MIC values of polymer 1c and AgPTS are 10 μg/mL and 39 μg/mL respectively, whereas the MIC value for the 1:0.5 composite is (5+2.5) μg/mL against S. aureus. The composite is particularly highly active against Gram-negative bacteria such as E. coli. For example, the 1:0.5 composite is active at (5+2.5) μg/mL against both these pathogens whereas the MIC values for the 1c and AgPTS are 312 μg/mL and 10 μg/mL against E. coli respectively. Effectiveness of these composites is further emphasized by the activity shown against various drug-resistant superbugs such as VRE, MRSA and K. pneumoniae. For example, the MIC values of the 1:0.5 composite are (5+2.5) μg/mL against both MRSA and VRE and (10+5) μg/mL against K. pneumoniae.

### Example 32: Antibacterial Kinetics of the Nanocomposites

To establish how fast these composites kill bacteria upon contact and also to find whether the polymeric composites act synergistically, the rate of antibacterial action was investigated towards S. aureus (FIG. 14). 96-Well plate was coated with the polymer, AgPTS and polymeric nanocomposite (1c: AgPTS=1:0.5), all at two different concentrations: MIC and 6×MIC. Antibacterial kinetics assay was performed similarly as mentioned earlier. The polymeric nanocomposite was found to kill bacteria at a much faster rate as compared to the polymer and AgPTS. Polymeric nanocomposite killed S. aureus (˜6 log reduction) at 90 min at 6×MIC whereas 1c killed S. aureus (˜6 log reduction) at 240 min at the same concentration (FIG. 14). AgPTS, on the other hand, was found to be bacteriostatic even at 6×MIC. These results thus indicated that the polymeric nanocomposite not only killed bacteria at faster rate but also acted synergistically.

### ADVANTAGE

The above mentioned implementation examples as described on this subject matter and its equivalent thereof have many advantages, including those which are described.

The disclosed compounds and/or derivatives in the present disclosure are completely insoluble in water and highly soluble in organic solvents. The organic solutions of these derivatives can be easily coated to prepare microbicidal paint. The compounds of the present disclosure show high antibacterial activity against various pathogens including drug resistant bacteria.

Although the subject matter has been described in considerable detail with reference to certain embodiments thereof, other embodiments are possible. As such, the spirit and scope of the invention should not be limited to the description of the embodiment contained herein.

1. A compound of Formula I 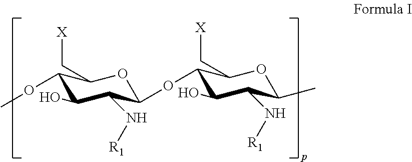
wherein:
X is 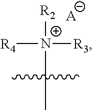
OH and combinations thereof;
R₂, R₃ and R₄ are independently selected from the group consisting of hydrogen, substituted or unsubstituted C₁₋₂₂ alkyl, substituted or unsubstituted C₆₋₁₀ aryl, 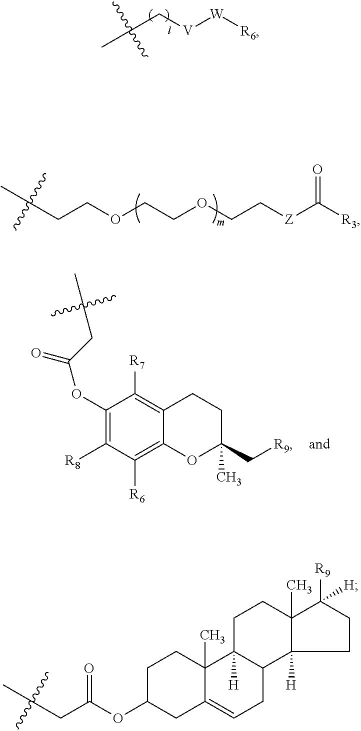
or
R₂ and R₃ taken together to form a substituted or unsubstituted cyclic ring system which is saturated or partially unsaturated and optionally have additional heteroatoms selected from O, N or S; or
R₂ and R₃ taken together to form a substituted or unsubstituted aromatic ring system optionally having heteroatoms selected from O, N or S; or
R₂, R₃ and R₄ may combine to form a substituted or unsubstituted bicylic ring system which is saturated, partially unsaturated or fully unsaturated, a substituted or unsubstituted aromatic ring system and optionally having heteroatoms selected from O, N or S;
V and W are independently selected from the group consisting of O, NH and —CO;
Z is O or —NH;
R₁ is selected from the group consisting of hydrogen, C₁₋₁₆ alkyl, C₆₋₁₀ aryl, —COR₁₀, and combinations thereof;
R₅ and R₉ are independently selected from the group consisting of hydrogen, substituted or unsubstituted C₁₋₁₆ alkyl, substituted or unsubstituted C₂₋₂₄ alkenyl, substituted or unsubstituted C₆₋₁₀ aryl, and combinations thereof;
R₆, R₇ and R₈ are independently selected from hydrogen and methyl;
A^{⊖} is negatively charged counter anion;
R₁₀ is selected from the group consisting of C₁₋₁₆ alkyl and C₆₋₁₀ aryl, wherein alkyl and aryl are optionally substituted with halogen, alkyl, and aryl;
l is 0 to 4;
m is 0 to 3; and
p is 1 to 1000,
wherein the degree of substitution of R₁ with hydrogen, C₁₋₁₆ alkyl, C₆₋₁₀ aryl, or —COR₁₀ in the compound of formula I is in the range of 20-100%; and the degree of substitution of X with 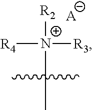
in the compound of formula I is in the range of 10-90%.

2. A compound of Formula I 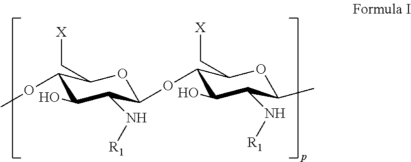
wherein:
X is 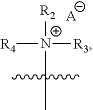
OH and combinations thereof;
R₂, R₃ and R₄ are independently selected from the group consisting of hydrogen, substituted or unsubstituted C₁₋₂₂ alkyl, substituted or unsubstituted C₆₋₁₀ aryl, 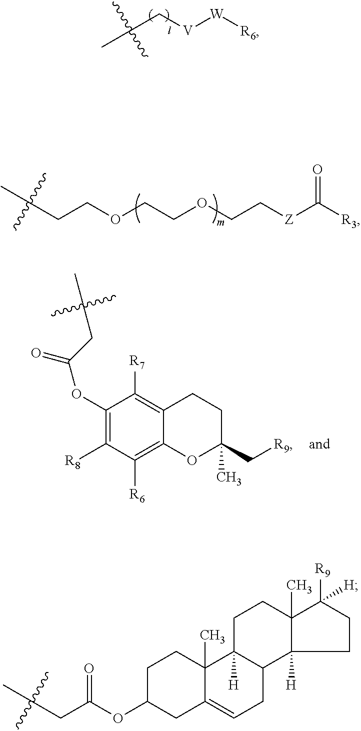
wherein alkyl, and aryl, are optionally substituted with one or more substituents selected from hydroxy, alkyl, aryl, alkoxy, halogen, haloalkyl, perhaloalkyl, cyano, OR₁₀, or
R₂ and R₃ taken together to form a substituted or unsubstituted cyclic ring system which is saturated or partially unsaturated and optionally having heteroatoms selected from O, N or S; or
R₂ and R₃ taken together to form a substituted or unsubstituted aromatic ring system optionally having heteroatoms selected from O, N or S and R₄ is absent; or
R₂, R₃ and R₄ may combine to form a substituted or unsubstituted bicylic ring system which is saturated, partially unsaturated or fully unsaturated, a substituted or unsubstituted aromatic ring system and optionally having heteroatoms selected from O, N or S; wherein the cyclic ring system, the aromatic ring system and the bicyclic ring system is further optionally substituted with 1 to 4 substituents independently selected from halo, alkyl, alkenyl, alkynyl, nitro, cyano, cycloalkyl, cycloalkylalkyl, aryl, arylalkyl, heterocyclyl, heterocyclylalkyl, heteroaryl, heteroarylalkyl and a compound of Formula II; 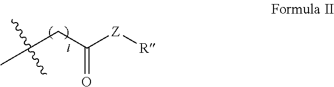
wherein the alkyl, aryl, heteroaryl is further optionally substituted with alkyl, cycloalkyl, cycloalkylalkyl, aryl, arylalkyl, heterocyclyl, heterocyclylalkyl, heteroaryl, heteroarylalkyl and a compound of Formula II,
V and W are independently selected from the group consisting of O, NH and —CO;
Z is O or NH;
R″ is selected from the group consisting of C₁₋₂₂ alkyl, or C₂₋₂₄ alkenyl;
R₁ is selected from the group consisting of hydrogen, C₁₋₁₆ alkyl, C₆₋₁₀ aryl, —COR₁₀, and combinations thereof;
R₅ and R₉ are independently selected from the group consisting of hydrogen, C₁₋₁₆ alkyl, C₂₋₂₄ alkenyl, C₆₋₁₀ aryl, and combinations thereof;
R₆, R₇ and R₈ are independently selected from hydrogen and methyl;
A^{⊖} is negatively charged counter anion;
R₁₀ is selected from the group consisting of C₁₋₁₆ alkyl and C₆₋₁₀ aryl, wherein alkyl and aryl are optionally substituted with halogen, alkyl, and aryl;
l is 0 to 4;
m is 0 to 3; and
p is 1 to 1000,
wherein the degree of substitution of R₁ with hydrogen, C₁₋₁₆ alkyl, C₆₋₁₀ aryl, or —COR₁₀ in the compound of formula I is in the range of 20-100%; and the degree of substitution of X with 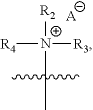
in the compound of formula I is in the range of 10-90%.

3. The compound of formula I as claimed in claim 1, wherein A^{⊖} is negatively charged counter anion selected from the group consisting of Cl⁻, Br⁻, I⁻, OH⁻, HCO₃⁻, CO₃²⁻, R₁₁COO⁻, R₁₁SO₄⁻, and R₁₁SO₃⁻, wherein R₁₁ is selected from the group consisting of hydrogen, C₁₋₆ alkyl and C₆₋₁₀ aryl, wherein alkyl and aryl are optionally substituted with hydroxyl, nitro, halogen, ester, alkyl, and aryl.

4. The compound of formula I as claimed in claim 1, wherein X with 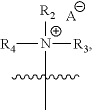
is selected from the group consisting of 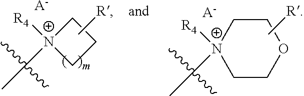
R₄ is selected from the group consisting of hydrogen, substituted or unsubstituted C₁₋₂₂ alkyl, substituted or unsubstituted C₆₋₁₀ aryl, 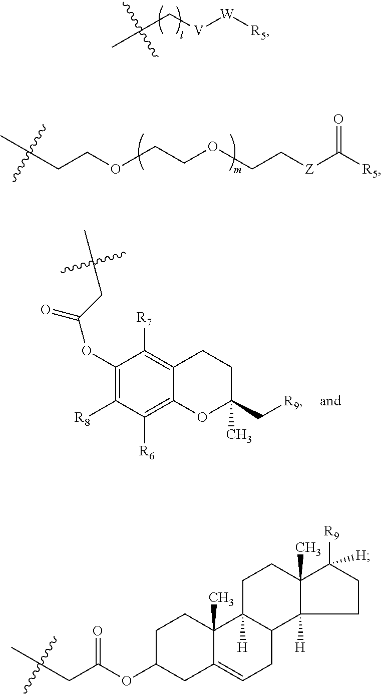
wherein alkyl, and aryl, are optionally substituted with one or more substituents selected from hydroxy, alkyl, aryl, alkoxy, halogen, haloalkyl, perhaloalkyl, cyano, —OR₁₀,
R′ is selected from the group consisting of alkyl, cycloalkyl, cycloalkylalkyl, aryl, arylalkyl, heterocycyl, heterocyclylalkyl, heteroaryl, heteroarylalkyl and a compound of Formula II; 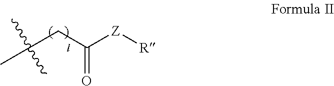
Z is O or NH;
R″ is selected from the group consisting of C₁₋₂₂ alkyl, or C₂₋₂₄ alkenyl;
A^{⊖} is negatively charged counter anion selected from the group consisting of Cl⁻, Br⁻, I⁻, OH⁻, HCO₃⁺, CO₃²⁻, R₁₁COO⁻, R₁₁SO₄⁻, and R₁₁SO₃⁻;
V and W are independently selected from the group consisting of O, NH and —CO;
R₅ and R₉ are independently selected from the group consisting of hydrogen, C₁₋₆ alkyl, C₂₋₂₄ alkenyl, C₆₋₁₀ aryl, and combinations thereof;
R₆, R₇ and R₈ are independently selected from hydrogen and methyl;
R₁₀ is selected from the group consisting of C₁₋₁₆ alkyl and C₆₋₁₀ aryl, wherein alkyl and aryl are optionally substituted with halogen, alkyl, and aryl;
R₁₁ is selected from the group consisting of hydrogen, C₁₋₆ alkyl and C₆₋₁₀ aryl, wherein alkyl and aryl are optionally substituted with hydroxyl, nitro, halogen, ester, alkyl, and aryl;
l is 0 to 4; and
m is 0 to 3.

5. The compound of formula I as claimed in claim 1, wherein X with 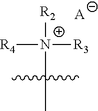
is selected from the group consisting of 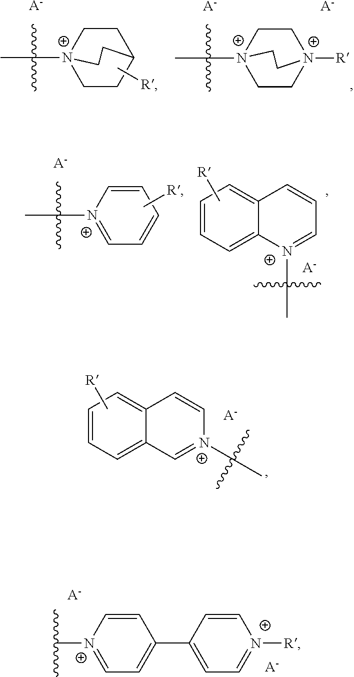
R′ is selected from the group consisting of alkyl, cycloalkyl, cycloalkylalkyl, aryl, arylalkyl, heterocyclyl, heterocyclylalkyl, heteroaryl, heteroarylalkyl and a compound of Formula II; 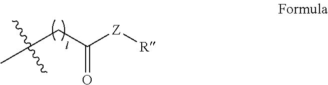
Z is O or NH;
R″ is selected from the group consisting of C₁₋₂₂ alkyl, or C₂₋₂₄ alkenyl;
A^{⊖} is negatively charged counter anion selected from the group consisting of Cl⁻, Br⁻, I⁻, OH⁻, HCO₃⁻, CO₃²⁻, R₁₁COO⁻, R₁₁SO₄⁻, and R₁₁SO₃⁻;
R₁₁ is selected from the group consisting of hydrogen, C₁₋₆ alkyl and C₆₋₁₀ aryl, wherein alkyl and aryl are optionally substituted with hydroxyl, nitro, halogen, ester, alkyl, and aryl;
l is 0 to 4; and
m is 0 to 3.

6. The compound of Formula I as claimed in claim 1, wherein R₂, R₃, and R₄ are independently selected from the group consisting of 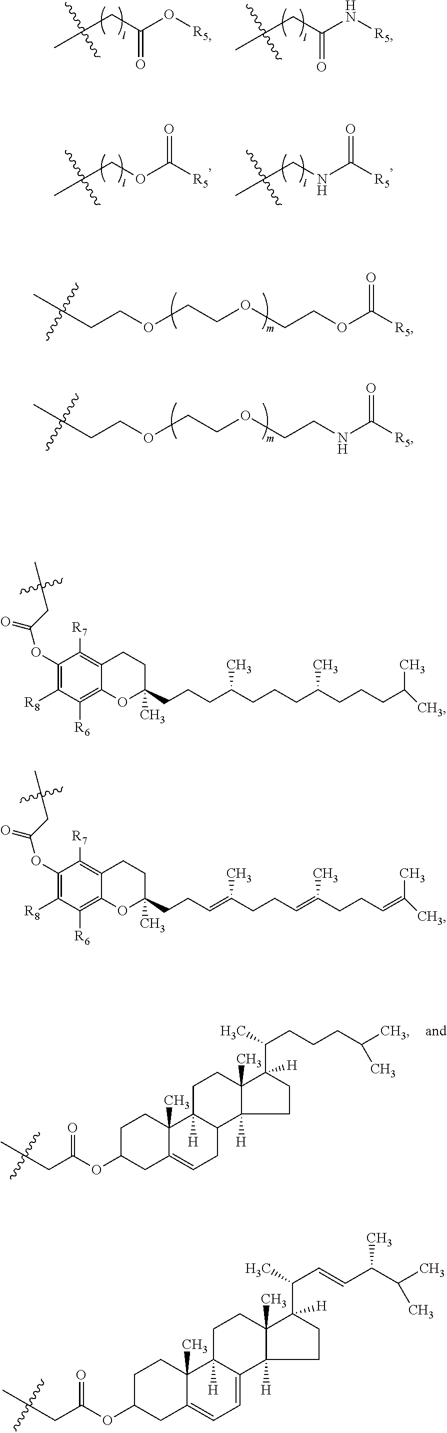
R₅ is selected from the group consisting of hydrogen, C₁₋₁₆ alkyl, C₂₋₂₄ alkenyl, C₆₋₁₀ aryl, and combinations thereof;
R₆, R₇ and R₈ are independently selected from hydrogen and methyl;
l is 0 to 4; and
m is 0 to 3.

7. The compound of formula I as claimed in claim 1, wherein R₂ and R₃ are independently selected from the group consisting of hydrogen, and C₁₋₂ alkyl;
R₄ is C₁₋₂₀ alkyl;
R₁ is independently selected from the group consisting of hydrogen, —COR₁₀, and combinations thereof;
A^{⊖} is selected from the group consisting of Cl⁻, Br⁻, R₁₁SO₃⁻;
R₁₀ is selected from the group consisting of C₁₋₁₆ alkyl and C₆₋₁₀ aryl, wherein alkyl and aryl are optionally substituted with halogen, alkyl, and aryl;
R₁₁ is selected from the group consisting of hydrogen, C₁₋₆ alkyl and C₆₋₁₀ aryl, wherein alkyl and aryl are optionally substituted with hydroxyl, nitro, halogen, ester, alkyl, and aryl,
wherein the degree of substitution of R₁ with —COR₁₀ in the compound of Formula I is in the range of 30-100%; and the degree of substitution of X with 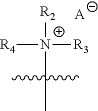
in the compound of Formula I is in the range of 20-80%.

8. The compound of Formula I as claimed in claim 1, wherein R₂ and R₃ are independently methyl;
R₄ is C₁₂₋₁₆ alkyl;
A^{⊖} is 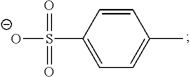
p is 500 to 900;
wherein the degree of substitution of X with 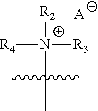
in the compound of Formula I is in the range of 40-70%.

9. The compound of formula I, as claimed in claim 1,
wherein:
X is a combination of 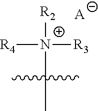
and OH;
R₂ and R₃ is methyl;
R₁ is —COCH₃;
R₄ is C₁₂-C₁₆ alkyl;
p is an integer 700-800;
A^{⊖} is 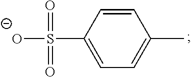
wherein the degree of substitution of X with 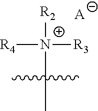
in the compound of Formula I is in the range of 40-70%.

10. The compound as claimed in claim 1 for use in antimicrobial coatings.

11. The compound as claimed in claim 10, wherein the coating is done by spin coating, brush coating, dip coating or painting.

12. The compound as claimed in claim 1 for use as antibacterial agents in the treatment of diseases caused by bacteria, fungi, and virus.

13. The compound as claimed in claim 1 for use as antibacterial agents in the treatment of diseases caused by Gram-positive and Gram-negative bacteria.

14. An article comprising a substrate, wherein the substrate is coated with or impregnated with the composition comprising the compound of claim 1, or the pharmaceutically acceptable salt.

15. A pharmaceutical composition comprising a compound as claimed in claim 1 with a pharmaceutically acceptable carrier, optionally in combination with one or more other pharmaceutical compositions.

16. A method of preparing biodegradable antimicrobial coatings and/or surfaces with or without pharmaceutical compositions.

17. The method as claimed in claim 16, wherein the surface is formed from material selected from the group consisting of metals, ceramics, glass, polymers, plastics, fibers and combinations thereof.

18. The method as claimed in claim 16, wherein the surface is the surface of a toy, bathroom fixture, countertop, tabletop, handle, computer, military gear, clothing, paper product, window, door, or interior wall fabric, gauze, tissue, surgical drape, air-filter, tubing, surgical instruments, device or implants to be placed into the body or tissue.

19. A process of preparing a compound of Formula I as claimed in claim 1, the process comprising:
(a) contacting a compound of Formula III, wherein R₁ and p are defined as above, 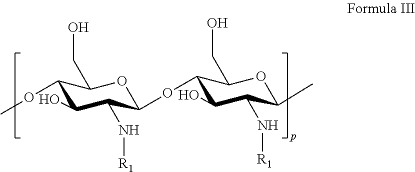
with R₁₁SO₃Cl, wherein R₁₁ is defined as above; in 0-10% wt/vol of lithium chloride and a solvent to obtain a compound of Formula IV, wherein R₁ and p are defined as above; Y is a combination of R₁₁SO₃— and OH,
wherein the degree of substitution of Y with R₁₁SO₃— in the compound of Formula IV is in the range of 30-90%;
with R₁₁SO₃— group at the C-6 position of Formula III. 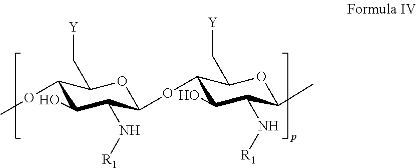
(b) reacting the compound of Formula IV with an acetylating agent in presence of a solvent to obtain an acetylated compound;
(c) treating the acetylated compound with a base to obtain O-deacetylated and N-acetalylated compound;
(d) contacting the O-deacetylated and N-acetalylated compound with NR₂R₃R₄, wherein R₂, R₃ and R₄ are defined as above, in presence of a solvent to obtain a solution; and
(e) cooling and precipitating the solution by a solvent to obtain a compound of Formula I.
wherein the degree of substitution of R₁ with hydrogen, C₁₋₁₆ alkyl, C₆₋₁₀ aryl, or —COR₁₀ in the compound of Formula I is in the range of 20-100%; and the degree of substitution of X with 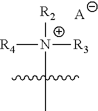
in the compound of Formula I is in the range of 10-90%.

20. The process as claimed in claim 19, wherein the solvent is selected from the group consisting of a polar solvent, non-polar solvent and mixtures thereof, preferably selected from the group consisting of N,N-dimethylformamide, N,N-dimethylacetamide, N,N-dimethylsulfoxide, N-methyl-2-pyrrolidone, pyridine, acetonitrile, acetone, dichloromethane, chloroform, 1,2-dichloroethane, methanol and mixtures thereof, preferably N,N-dimethylacetamide.

21. The process as claimed in claim 20, the non-polar solvent is selected from the group consisting of tetrahydrofuran, hexane, pentane, benzene, toluene and mixtures thereof.

22. The process as claimed in claim 19, wherein the acetylating agent is selected from the group consisting of acetic anhydride, acetyl chloride, preferably acetic anhydride.

23. The process as claimed in claim 19, wherein the base is selected from the group consisting of potassium hydroxide, sodium hydroxide, barium hydroxide, cesium hydroxide, strontium hydroxide, calcium hydroxide, lithium hydroxide, and rubidium hydroxide preferably potassium hydroxide.

24. A process of preparing a compound of Formula I as claimed in claim 1, the process comprising:
(a) contacting a compound of Formula III, wherein R₁ is independently selected from the group consisting of hydrogen, —COR₁₀, and combinations thereof; R₁₀ is C₁ alkyl; and p is 700 to 800, 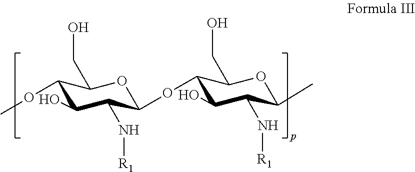
with R₁SO₃Cl, wherein R₁₁ is C₆ aryl, wherein aryl is substituted with alkyl; in 5% wt/vol of lithium chloride-N,N-dimethylacetamide solvent system to obtain a compound of Formula IV with R₁₁SO₃— group at the C-6 position of Formula III.
wherein R₁ is independently selected from the group consisting of hydrogen, —COR₁₀, and combinations thereof; R₁₀ is C₁ alkyl; Y is a combination of R₁₁SO₃— and OH, and p is 700 to 800.
wherein the degree of substitution of Y with R₁₁SO₃— in the compound of Formula IV is in the range of 30-90%; 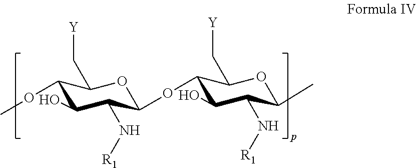
(b) reacting the compound of Formula IV with an acetic anhydride in presence of methanol to obtain an acetylated compound;
(c) treating the acetylated compound with a methanolic potassium hydroxide to obtain O-deacetylated and N-acetalylated compound;
(d) contacting the O-deacetylated and N-acetalylated compound with NR₂R₃R₄ selected from the group consisting of N,N-dimethyl dodecylamine, N,N-dimethyl tetradecylamine or N,N-dimethyl hexadecylamine in presence of a solvent selected from N,N-dimethyl acetamide or N,N-dimethyl sulfoxide to obtain a solution;
(e) cooling and precipitating the solution by a solvent selected from the group consisting of diethylether, n-hexane, acetone and combinations thereof to obtain a compound of Formula I.
wherein the degree of substitution of R₁ with hydrogen, C₁₋₁₆ alkyl, C₆₋₁₀ aryl, or —COR₁₀ in the compound of Formula I is in the range of 30-100%; and the degree of substitution of X with 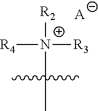
in the compound of Formula I is in the range of 40-70%.

25. A process of making nanocomposites by using any compound as claimed in claim 1, the process comprising
(a) dissolving a compound of Formula I in an organic solvent;
(b) adding to a solution of silver slat of formula R-M in another organic solvent; and
(c) keeping the mixture at room temperature for 6-72 h.

26. The process as claimed in claim 25, wherein the R is selected from the group consisting of NO₃⁻, Cl⁻, R′COO—, R′SO₃—, R′SO₂N—;
wherein R′ is selected from the group consisting of C₁₋₁₆ acyclic or cyclic alkyl and C₆₋₁₀ aryl, wherein alkyl and aryl are optionally substituted with halogen, alkyl, and aryl;
M is selected from the group of silver, or gold, preferably silver.

27. The process as claimed in claim 25, wherein the organic solvent is selected from the group consisting of N,N-dimethylformamide, N,N-dimethylacetamide, dimethylsulfoxide, N-methyl-2-pyrrolidone, acetone, methanol, ethanol, water and combinations thereof, preferably methanol and dimethyl sulfoxide.

28. The nanocomposite as claimed in claim 25 for use as antibacterial agents in the treatment of diseases caused by bacteria, fungi, and virus.

29. The nanocomposite as claimed in claim 25 for use in antimicrobial coatings.

30. An article comprising a substrate, wherein the substrate is coated with or impregnated with the composition comprising the nanocomposite of claim 25, or the pharmaceutically acceptable salt.

31. A pharmaceutical composition comprising a composite as claimed in claim 25 with a pharmaceutically acceptable carrier, optionally in combination with one or more other pharmaceutical compositions.
